(19) United States
(12) Patent Application Publication
(10) Pub. No.: US 2026/0123897 A1
(43) Pub. Date: May 7, 2026

(54) SYSTEM AND METHOD FOR MEDICAL IMAGING

(71) Applicant: SHANGHAI UNITED IMAGING HEALTHCARE CO., LTD., Shanghai (CN)

(72) Inventors: Tong LI, Shanghai (CN); Tao CHEN, Shanghai (CN); Beien WANG, Shanghai (CN); Lei SHI, Shanghai (CN)

(73) Assignee: SHANGHAI UNITED IMAGING HEALTHCARE CO., LTD., Shanghai (CN)

(21) Appl. No.: 19/379,658

(22) Filed: Nov. 4, 2025

(30) Foreign Application Priority Data
Nov. 4, 2024 (CN) 202411564551.0
Dec. 31, 2024 (CN) 202411998103.1
Dec. 31, 2024 (CN) 202411998774.8

(51) Int. Cl. A61B 6/03; A61B 5/11; A61B 6/00; A61B 6/40

(52) U.S. Cl. CPC A61B 6/037; A61B 5/1128; A61B 6/4014; A61B 6/4458; A61B 6/4464; A61B 6/5217

(57) ABSTRACT

Embodiments of the present disclosure provide a system and a method for medical imaging. The system includes at least one storage device including a set of instructions, and at least one processor configured to communicate with the at least one storage device. When executing the set of instructions, the at least one processor is configured to direct the system to perform operations including: obtaining first image data of the subject acquired by one or more first imaging devices; determining, based on the first image data of the subject, pose information of the subject; determining, based on the pose information of the subject, reference motion information of each of at least one of one or more detectors of a second imaging device; and controlling a movement of each of the at least one of the one or more detectors based on the reference motion information for the second imaging device acquiring second image data of the subject.

### CROSS-REFERENCE

The present disclosure claims priority to Chinese Application No. 202411998103.1 filed on Dec. 31, 2024, Chinese Application No. 202411564551.0 filed on Nov. 4, 2024, and Chinese Application No. 202411998774.8 filed on Dec. 31, 2024, the entire contents of each of which are hereby incorporated by reference.

### TECHNICAL FIELD

The present disclosure relates to the field of medical scanning, particularly regarding a system and a method for medical imaging.

### BACKGROUND

Single-Photon Emission Computed Tomography (SPECT) is a nuclear medicine imaging technique widely used in clinical diagnostics, particularly in cardiology, oncology, neurology, and skeletal disorders. It generates tomographic images by detecting gamma rays emitted from radiopharmaceuticals within the body, thereby assessing organ function, blood flow, and pathological conditions. Before a SPECT scan, a motion path of a detector of the SPECT needs to be determined and then the detector may be controlled to be moved according to the determined motion path.

Therefore, it is desired to provide a method and a system for motion path planning for a detector of an imaging system with improved efficiency and accuracy.

### SUMMARY

One or more embodiments of the present disclosure provide a system. The system includes at least one storage device including a set of instructions, and at least one processor configured to communicate with the at least one storage device. When executing the set of instructions, the at least one processor is configured to direct the system to perform operations including: obtaining first image data of a subject acquired by one or more first imaging devices; determining, based on the first image data of the subject, pose information of the subject; determining, based on the pose information of the subject, reference motion information of each of at least one of one or more detectors of a second imaging device; and controlling a movement of each of the at least one of the one or more detectors based on the reference motion information for the second imaging device acquiring second image data of the subject.

In some embodiments, the first image data of the subject is acquired by the one or more first imaging devices during a scan of the subject or before the scan of the subject.

In some embodiments, determining, based on the pose information of the subject, reference motion information of each of at least one of one or more detectors of a second imaging device includes: obtaining initial motion information based on a first portion of one or more scanning parameters and the pose information; and determining the reference motion information by optimizing the initial motion information based on a second portion of the one or more scanning parameters.

In some embodiments, determining the reference motion information by optimizing the initial motion information based on a second portion of the one or more scanning parameters includes: determining one or more optimization objectives based on the second portion of the one or more scanning parameters; and determining the reference motion information by optimizing the initial motion information based on the one or more optimization objectives using an optimization model, wherein the reference motion information satisfies the one or more optimization objectives.

In some embodiments, the optimization model is constructed based on an objective function, one or more constraints on at least one of reference path parameters of the reference motion information, the objective function being constructed based on the one or more optimization objectives and initial path parameters of the initial motion information, and the optimizing the initial motion information based on the one or more optimization objectives using an optimization model includes: optimizing the initial motion information by performing an iterative process based on the objective function, one or more constraints, and one or more optimization objectives to adjust the initial motion information.

In some embodiments, the optimization model is a first trained machine learning model, and optimizing the initial motion information based on the one or more optimization objectives includes: inputting the initial motion information and the one or more optimization objectives into the first trained machine learning model to generate the reference motion information.

In some embodiments, determining, based on the pose information of the subject, reference motion information of each of at least one of one or more detectors of a second imaging device includes: determining, based on a scanning range of the subject and the pose information, a motion range of the each of at least one of one or more detectors; and determining the reference motion information based on the motion range.

In some embodiments, determining, based on the pose information of the subject, reference motion information of each of at least one of one or more detectors of a second imaging device includes: determining, based on the pose information of the subject, the reference motion information using a second trained machine learning model.

In some embodiments, an input of the second trained machine learning model includes the pose information of the subject and at least one of medical image data acquired by a third imaging device, physiological status information of the subject, or one or more scanning parameters of the subject, wherein the physiological status information of the subject is determined using the first imaging device.

In some embodiments, a count of the one or more detectors exceeds 1, and at least a portion of the one or more detectors are arranged along an axis of the second scanning device.

In some embodiments, controlling a movement of each of the at least one of the one or more detectors based on the reference motion information for the second imaging device acquiring second image data of the subject includes: controlling the one or more detectors to move simultaneously based on the reference motion information of each of the one or more detectors to acquire the second image data of different portions of the subject during a same time period, and the operations further include determining target image data of the subject based on the second image data of the different portions of the subject.

In some embodiments, the second imaging device includes multiple imaging modules, each of the multiple imaging modules includes at least one of the one or more detectors and a robot joint assembly for providing multiple movement freedoms of the one of the one or more detectors, and each of the multiple imaging modules includes a first controller configured to control a component of the robot joint assembly to move based on the reference motion information.

In some embodiments, at least one of the one or more detectors is detachably connected with the second imaging device, and one of the one or more first imaging devices is arranged on one of the one or more detectors or on a ceiling of a scanning room where the second imaging device is located.

In some embodiments, the second imaging device includes multiple imaging modules, each of the multiple imaging modules includes one of the one or more detectors, a first controller, and a robot joint assembly for providing multiple movement freedoms of the one of the one or more detectors, the second imaging device further includes a second controller in communication with the first controller of each of the multiple imaging modules, wherein the second controller is configured to determine the reference motion information of each of the one or more detectors based on the pose information, the one or more scanning parameters, and position information of the one or more detectors, and transmit the reference motion information of each of the one or more detectors to a corresponding first controller.

In some embodiments, the pose information of the subject is denoted by a first contour of the subject, and determining, based on the pose information of the subject, reference motion information of each of at least one of one or more detectors of a second imaging device includes: obtaining a second contour of a couch where the subject is located; determining a third contour by combining the first contour and the second contour; and determining the reference motion information based on the third contour.

In some embodiments, determining the reference motion information based on the third contour includes determining a minimum convex hull of the third contour and determining the reference motion information based on the minimum convex hull.

In some embodiments, determining, based on the first image data of the subject, pose information of the subject includes: controlling the one of the one or more detectors to move along an axis of the second imaging device for one of the one or more first imaging device arranged on the one of the one or more detectors acquiring first sub-image data is at each of different positions; determining a first sub-contour of a portion of the subject based on the first sub-image data of the subject; and determining the first contour based on first sub-contours of different portions of the subject.

In some embodiments, the second contour includes a first sub-contour and a second sub-contour associated with a surface of the couch where the subject is located, and determining a third contour by combining the first contour and the second contour includes: combining the first sub-contour of the second contour and the first contour via the second sub-contour of the second contour.

One or more embodiments of the present disclosure provide a method implemented on a computing device having at least one processor and at least one storage medium including a set of instructions for processing service requests received from a requester terminal. The method includes: obtaining first image data of a subject acquired by one or more first imaging devices; determining, based on the first image data of the subject, pose information of the subject; determining, based on the pose information of the subject, reference motion information of each of at least one of one or more detectors of a second imaging device; and controlling a movement of each of the at least one of the one or more detectors based on the reference motion information for the second imaging device to acquire second image data of the subject.

One or more embodiments of the present disclosure provide a non-transitory computer readable medium. The non-transitory computer readable medium includes executable instructions that, when executed by at least one processor, direct the at least one processor to perform a method including: obtaining first image data of a subject acquired by one or more first imaging devices; determining, based on the first image data of the subject, pose information of the subject; determining, based on the pose information of the subject, reference motion information of each of at least one of one or more detectors of a second imaging device; and controlling a movement of each of the at least one of the one or more detectors based on the reference motion information for the second imaging device to acquire second image data of the subject.

### BRIEF DESCRIPTION OF THE DRAWINGS

The present disclosure may be further described in terms of exemplary embodiments, which may be described in detail with reference to the drawings. These embodiments are not limiting, and in these embodiments, the same reference numerals in the various drawings represent similar structures, and where:

FIG. 1 is a schematic diagram illustrating an exemplary imaging system according to some embodiments of the present disclosure.

FIG. 2 is a schematic diagram illustrating exemplary an exemplary computing device on which the processing device or the terminal device may be implemented according to some embodiments of the present disclosure.

FIG. 3 is a schematic structural diagram of a second imaging device according to some embodiments of the present disclosure.

FIG. 4 is a flowchart of an exemplary process for a motion control of a detector according to some embodiments of the present disclosure.

FIG. 5 is a schematic structural diagram of a processing device according to some embodiments of the present disclosure.

FIG. 6 is a schematic diagram of a contour, a minimum convex hull, and a motion path according to some embodiments of the present disclosure.

FIG. 7 is a schematic diagram of an installation position of a first imaging device according to some embodiments of the present disclosure.

FIG. 8 is a schematic diagram of an installation position of a first imaging device according to some embodiments of the present disclosure.

FIG. 9 is a schematic diagram of splicing a top surface contour of a subject and a bottom surface contour of a couch according to some embodiments of the present disclosure.

FIG. 10 is a schematic diagram of an exemplary detector distribution in an imaging system according to some embodiments of the present disclosure.

FIG. 11 is a schematic diagram of another exemplary detector distribution in an imaging system according to some embodiments of the present disclosure.

FIG. 12 is a schematic diagram of another exemplary detector distribution in an imaging system according to some embodiments of the present disclosure.

FIG. 13 is a schematic diagram of determining an overall image based on local images of different portions according to some embodiments of the present disclosure.

FIG. 14 is a schematic diagram of determining an overall image based on local images of different portions according to other embodiments of the present disclosure.

FIG. 15 is a flowchart of an exemplary process for an imaging way of an imaging system according to some embodiments of the present disclosure.

FIG. 16 is a flowchart of an exemplary process for scanning the subject according to some embodiments of the present disclosure.

FIG. 17 is a schematic diagram of a second imaging device according to some embodiments of the present disclosure.

### DETAILED DESCRIPTION

To more clearly illustrate the technical solutions of the embodiments of the present disclosure, the accompanying drawings required to be used in the description of the embodiments will be briefly described below. Obviously, the accompanying drawings in the following description are only some examples or embodiments of the present disclosure, and a person of ordinary skill in the art may apply the present disclosure to other similar scenarios in accordance with these drawings without creative labor. The present disclosure may be applied to other similar scenarios based on these drawings without creative labor. Unless obviously obtained from the context or the context illustrates otherwise, the same numeral in the drawings refers to the same structure or operation.

It should be understood that the terms “system,” “device,” as used herein, “unit,” and/or “module” as used herein are a way to distinguish between different components, elements, parts, sections or assemblies at different levels. However, said words may be replaced by other expressions if other words accomplish the same purpose.

As shown in the disclosure and claims, unless the context clearly indicates an exception, words such as “one,” “a,” “an,” and/or “the” do not specifically refer to the singular, but may also include the plural. Generally, the terms “including,” and “comprising” suggest only the inclusion of clearly identified steps and elements. In general, the terms “including,” and “comprising” only suggest the inclusion of explicitly identified steps and elements that do not constitute an exclusive list, and the method or device may also include other steps or elements.

Flowcharts are used in the present disclosure to illustrate operations performed by a system according to embodiments of the present disclosure. It should be appreciated that the preceding or following operations are not necessarily performed in an exact sequence. Instead, steps may be processed in reverse order or simultaneously. Also, it is possible to add other operations to these processes or remove a step or steps from them.

Conventional SPECT systems typically employ single or dual detectors. When scanning different body parts, these detectors must operate sequentially. This step-by-step approach significantly prolongs scan duration and introduces potential inaccuracies due to subject movement or the need for repositioning between scans. Furthermore, traditional motion control for detectors often relies on optimizing transient velocity or dwell time at data collection points to match the expected radioactive emission distribution of a Region of Interest (ROI). These methods place high demands on algorithmic precision, involve complex and cumbersome implementation steps, and consequently make practical application of optimized detector path planning challenging.

Another approach to improve efficiency is continuous scanning, where the gantry and detectors rotate around the subject uninterrupted. While this increases the duty cycle of data acquisition, it requires accurate pre-acquisition of the subject's external contour. If SPECT itself is used to obtain this contour information, a scan covering at least one detector's axial dimension is necessary, inevitably exposing the subject to radiation beyond the diagnostically required range. This results in unnecessary radiation dose, posing a significant drawback.

Therefore, existing technologies in SPECT imaging grapple with key limitations: inefficient sequential scanning, complex and difficult-to-implement motion path planning, and the dilemma between scan efficiency and increased subject radiation exposure when attempting continuous scanning. An effective solution addressing these intertwined challenges of scanning efficiency, motion path planning complexity, and does reduction is presently lacking.

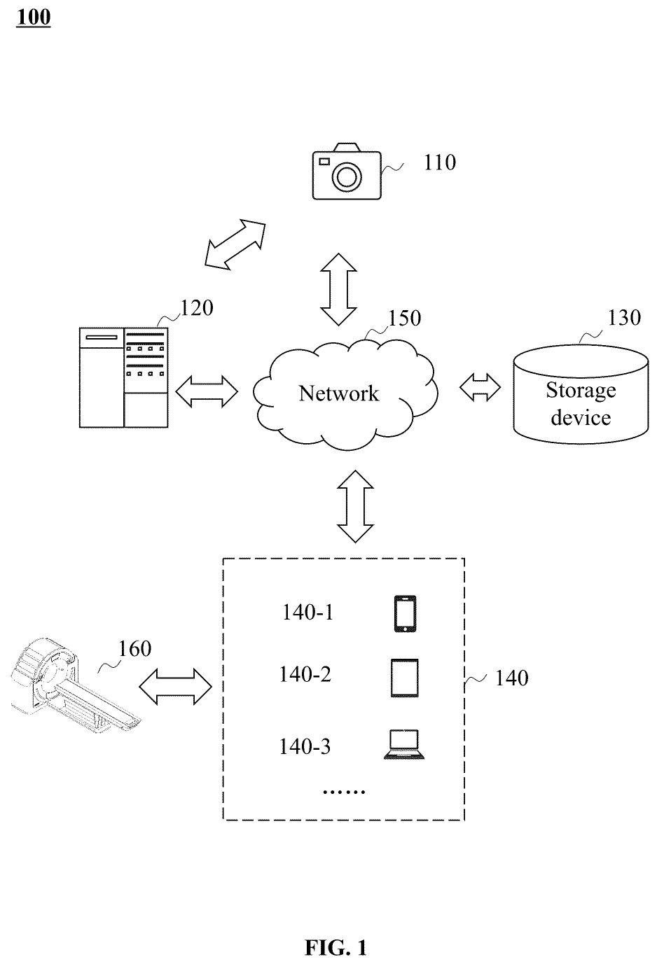

FIG. 1 is a schematic diagram illustrating an exemplary imaging system according to some embodiments of the present disclosure. As shown, the imaging system 100 may include one or more first imaging devices 110, a processing device 120, a storage device 130, one or more terminals 140, a network 150, and a second imaging device 160. The components in the imaging system 100 may be connected in one or more of various ways. Merely by way of example, as illustrated in FIG. 1, the first imaging devices 110 and/or the second imaging device 160 may be connected to the processing device 120 through the network 150. As another example, the first imaging devices 110 and/or the second imaging device 160 may be connected to the processing device 120 directly as indicated by the bi-directional arrow in dotted lines linking the first imaging devices 110 and the processing device 120. As a further example, the storage device 130 may be connected to the processing device 120 directly or through the network 150. As still a further example, one or more terminals 140 may be connected to the processing device 120 directly (as indicated by the bi-directional arrow in dotted lines linking the terminal(s) 140 and the processing device 120) or through the network 150.

The first imaging devices 110 may be configured to acquire first image data indicating and/or including appearance characteristic information of a subject (e.g., shape, color, texture, and/or spatial relationship). In some embodiments, the first image data may be configured to acquire the motion data of a subject. In some embodiments, the first image data may be used to distinguish different portions of the subject and/or distinguish the subject from other objects around the subject. In other words, the first imaging devices 110 may be used to identify and/or position different portions of the subject and/or identify and/or position the subject from other objects around the subject. For example, different portions of the subject and/or the subject may be identified from a video acquired by an optical sensor (e.g., a charge-coupled device). As another example, different portions of the subject and/or the subject may be identified from point cloud data acquired by a LiDAR sensor.

The one or more first imaging devices 110 may include one or more sensors. Exemplary sensors may include an optical sensor, a radar device (e.g., a LiDAR), a time-of-flight (TOF) device, a structured light scanner, a camera (e.g., a 2D camera, a 3D camera), or the like, or any combination thereof. For example, a sensor including an optical sensor, a camera (e.g., a digital camera, an analog camera, etc.), a red-green-blue (RGB) sensor, an RGB-depth (RGB-D) sensor, or another device that can capture optical image data of the subject. As another example, the sensor (e.g., a radar device, a structured light scanner, etc.) may be used to acquire point-cloud data of the subject. The point-cloud data may include a plurality of data points, each of which may represent a physical point on a body surface of the subject and can be described using one or more feature values of the physical point (e.g., feature values relating to the position and/or the composition of the physical point). As still another example, a sensor (e.g., a 3D camera) may be used to acquire depth image data of the subject. The depth image data may refer to image data that includes depth information of each physical point on the body surface of the subject, such as a distance from each physical point to a specific point (e.g., an optical center of the sensors). The 3D camera is a range sensing device, e.g., a structured light scanner, a time-of-flight (TOF) device, a stereo triangulation camera, a sheet of light triangulation device, an interferometry device, a coded aperture device, a stereo matching device, or the like, or any combination thereof.

The second imaging device 160 may generate or provide medical image data via scanning a subject or a part of the subject. The medical image data may represent the anatomical structure, physiological function, and/or metabolic activity of the subject. In some embodiments, the second imaging device 160 may be a medical imaging device, for example, a SPECT scanner, an X-ray radiation device (e.g., a digital subtraction angiography (DSA) scanner, a digital radiography (DR) scanner), or the like, or any combination thereof. In some embodiments, the second imaging device 160 may include a single-modality scanner. The single-modality scanner may include, for example, a SPECT scanner. In some embodiments, the second imaging device 160 may include a multi-modality scanner. In some embodiments, the multi-modality scanner may include a SPECT-X-ray scanner, etc. The multi-modality scanner may perform multi-modality imaging simultaneously.

In some embodiments, the subject may include a body, a substance, or the like, or any combination thereof. In some embodiments, the subject may include a specific portion of a body, such as a head, a thorax, an abdomen, an upper limb, a lower limb, or the like, or any combination thereof. In some embodiments, the subject may include a specific organ, such as an esophagus, a trachea, a bronchus, a stomach, a gallbladder, a small intestine, a colon, a bladder, a ureter, a uterus, a fallopian tube, a knee joint, an ankle joint, a thigh bone, a shin bone, etc. In some embodiments, the subject may include a physical model (also referred to as a mockup). The physical model may include one or more materials constructed as different shapes and/or dimensions. In the present disclosure, “object” and “subject” are used interchangeably. In some embodiments, the second imaging device 160 may include a scanning table. The subject may be placed on the scanning table for imaging.

In some embodiments, the second imaging device 160 may transmit the image data via the network 150 to the processing device 120, the storage device 130, and/or the terminal(s) 140. For example, the medical image data may be sent to the processing device 120 for further processing or may be stored in the storage device 130.

The processing device 120 may process data and/or information obtained from the first imaging device 110, the storage device 130, and/or the terminal(s) 140. For example, the processing device 120 may perform planning and adjustment of a motion path of the detector by processing the pose information of the subject input by the first imaging device 110 and a scan requirement input by a user, and output the motion path to a motion control system of the second imaging device 160. The motion control system of the second imaging device 160 controls the detector to move relative to the subject according to the motion path. Due to the introduction of the first imaging device, the system can obtain key information such as a distance between the subject and the detector, a motion speed of the detector, and the pose information of the subject in real time, thereby intelligently controlling the motion path of the detector.

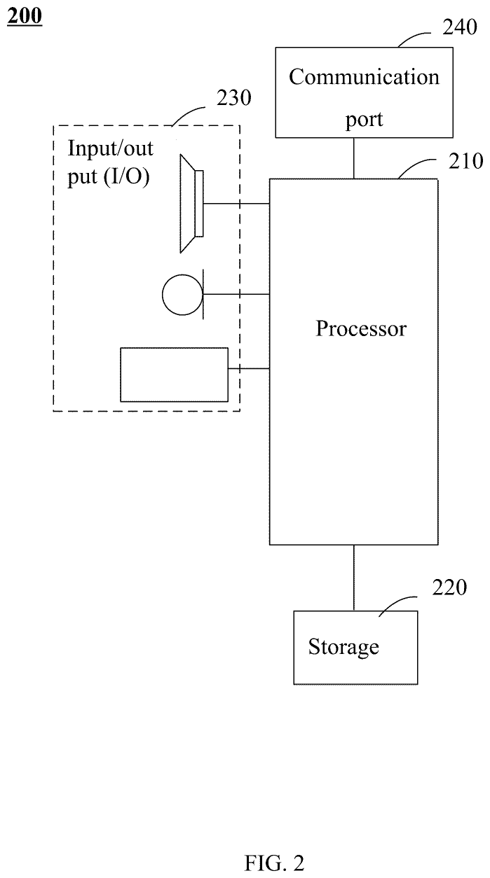

In some embodiments, the processing device 120 may be a single server or a server group. The server group may be centralized or distributed. In some embodiments, the processing device 120 may be local or remote. For example, the processing device 120 may access information and/or data from the first imaging devices 110, the storage device 130, and/or the terminal(s) 140 via the network 150. As another example, the processing device 120 may be directly connected to the first imaging devices 110, the terminal(s) 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be implemented by a computing device 200 having one or more components as described in connection with FIG. 2.

The storage device 130 may store data, instructions, and/or any other information. In some embodiments, the storage device 130 may store data obtained from the first imaging devices 110, the processing device 120, and/or the terminal(s) 140. In some embodiments, the storage device 130 may store data and/or instructions that the processing device 120 may execute or use to perform exemplary methods described in the present disclosure. In some embodiments, the storage device 130 may include a mass storage, a removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform as described elsewhere in the disclosure. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the imaging system 100 (e.g., the processing device 120, the terminal(s) 140, etc.). One or more components in the imaging system 100 may access the data or instructions stored in the storage device 130 via the network 150. In some embodiments, the storage device 130 may be part of the processing device 120.

The terminal(s) 140 may be connected to and/or communicate with the first imaging device 110, the processing device 120, and/or the storage device 130. For example, the terminal(s) 140 may obtain a processed image from the processing device 120. As another example, the terminal(s) 140 may obtain image data acquired by the first imaging device 110 and transmit the image data to the processing device 120 to be processed. In some embodiments, the terminal(s) 140 may include a mobile device 140-1, a tablet computer 140-2, a laptop computer 140-3, or the like, or any combination thereof. For example, the mobile device 140-1 may include a mobile phone, a personal digital assistance (PDA), a gaming device, a navigation device, a point of sale (POS) device, a laptop, a tablet computer, a desktop, or the like, or any combination thereof. In some embodiments, the terminal(s) 140 may include an input device, an output device, etc. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to the processing device 120 via, for example, a bus, for further processing. Other types of the input device may include a cursor control device, such as a mouse, a trackball, or cursor direction keys, etc. The output device may include a display, a speaker, a printer, or the like, or a combination thereof. In some embodiments, the terminal(s) 140 may be part of the processing device 120.

The network 150 may include any suitable network that can facilitate the exchange of information and/or data for the imaging system 100. In some embodiments, one or more components of the imaging system 100 (e.g., the first imaging device 110, the processing device 120, the storage device 130, the terminal(s) 140, etc.) may communicate information and/or data with one or more other components of the imaging system 100 via the network 150. For example, the processing device 120 may obtain image data from the first imaging device 110 via the network 150. As another example, the processing device 120 may obtain user instruction(s) from the terminal(s) 140 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the imaging system 100 may be connected to the network 150 to exchange data and/or information.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the storage device 130 may be a data storage including cloud computing platforms, such as, public cloud, private cloud, community, and hybrid clouds, etc. However, those variations and modifications do not depart from the scope of the present disclosure.

FIG. 2 is a schematic diagram illustrating exemplary an exemplary computing device on which the processing device or the terminal device may be implemented according to some embodiments of the present disclosure. As illustrated in FIG. 2, the computing device 200 may include a processor 210, a storage 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process image data obtained from the first imaging device 110, the terminal(s) 140, the storage device 130, and/or any other component of the Imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the first imaging devices 110, the second imaging device 160, the terminal(s) 140, the storage device 130, and/or any other component of the Imaging system 100. In some embodiments, the storage device 220 may store computer programs, such as software programs and modules of application software, such as the computer program corresponding to the motion control method of the detector in some embodiments of the present disclosure. The processor 210 executes various functional applications and data processing by running the computer program stored in the storage 220, thereby implementing the above motion control method.

In some embodiments, the storage 220 may include a mass storage, a removable storage, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure. For example, the storage 220 may store a program for the processing device 120 for determining reference motion information. In some instances, the storage 220 may further include memory remotely set relative to the processor 210, and these remote memories may be connected to the terminal through a network. Examples of the network include, but are not limited to, the Internet, an intranet, a local area network, a mobile communication network, and combinations thereof.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touch screen, a microphone, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touch screen, or the like, or a combination thereof. In one example, the I/O 230 may include a Network Interface Controller (NIC), which may be connected to other network devices through a base station to communicate with the Internet. In one example, the I/O 230 may be a Radio Frequency (RF) module, which is used to communicate with the Internet wirelessly.

The communication port 240 may be connected to a network (e.g., the network 150) to facilitate data communications. The communication port 240 may establish connections between the processing device 120 and the first imaging device 110, the terminal(s) 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMAX™ link, a WLAN link, a ZigBee link, a mobile network link (e.g., 3G, 4G, 5G, etc.), or the like, or any combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

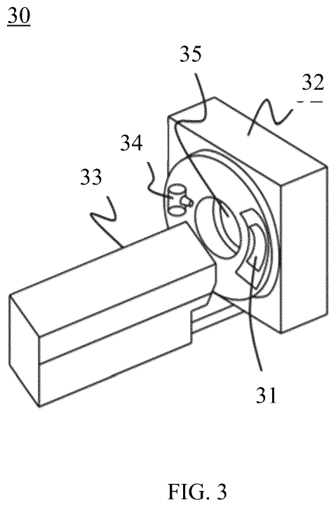

FIG. 3 is a schematic structural diagram of a second imaging device according to some embodiments of the present disclosure. The second imaging device may include a SPECT scanner, a DR scanner, a DSR scanner, etc. As shown in FIG. 3, the second imaging device 30 may include: one or more detectors 31, a gantry 32, a couch 33, a radiation source 34, and a detection area 35. The gantry 32 supports at least one of the detectors 31 to move (e.g., translate or rotate), so that the detector 31 may rotate around the subject by a certain angle (e.g., 50 degrees or 360 degrees) and/or translate along the axis of the second imaging device 30, and acquire a series of planar projection images from a plurality of angles and orientations. The couch 33 may be used for carrying the subject into the field of view of the SPECT detectors 31 for data acquisition and imaging.

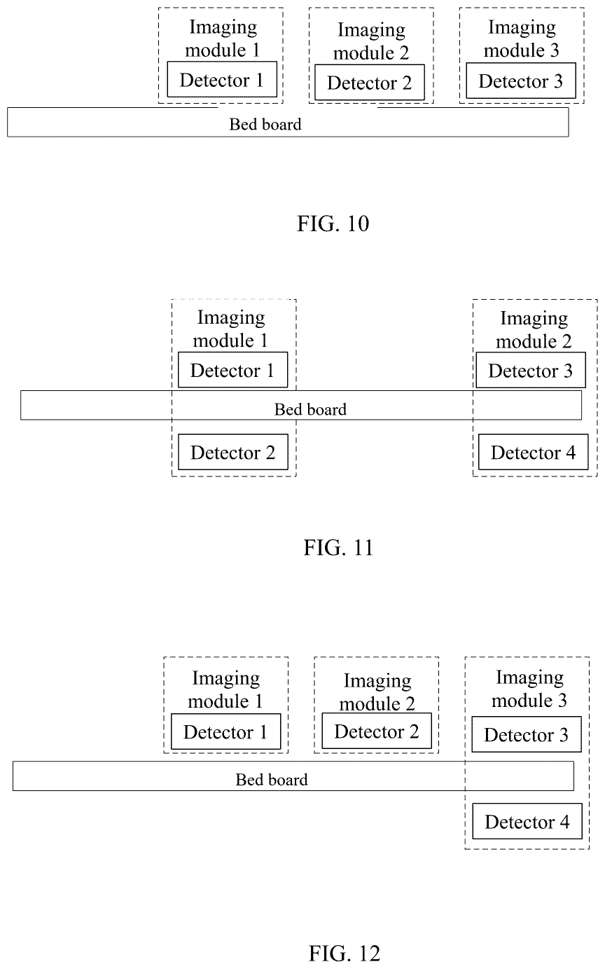

The one or more detectors 31 may be configured to detect radiation signals from the detection area 35 and generate medical image data. More descriptions regarding the one or more detectors may be found elsewhere in the present disclosure (e.g., FIGS. 10-12 and the descriptions thereof).

In some embodiments, the second imaging device may include a processing device (e.g., the processing device 120 as shown in FIG. 1). The processing device may specifically be a computer, including data acquisition software, display software, image processing software, dynamic image analysis software, etc., for reconstructing, correcting, analyzing, and displaying the acquired original images, and executing the motion control method provided by the embodiments of the present disclosure.

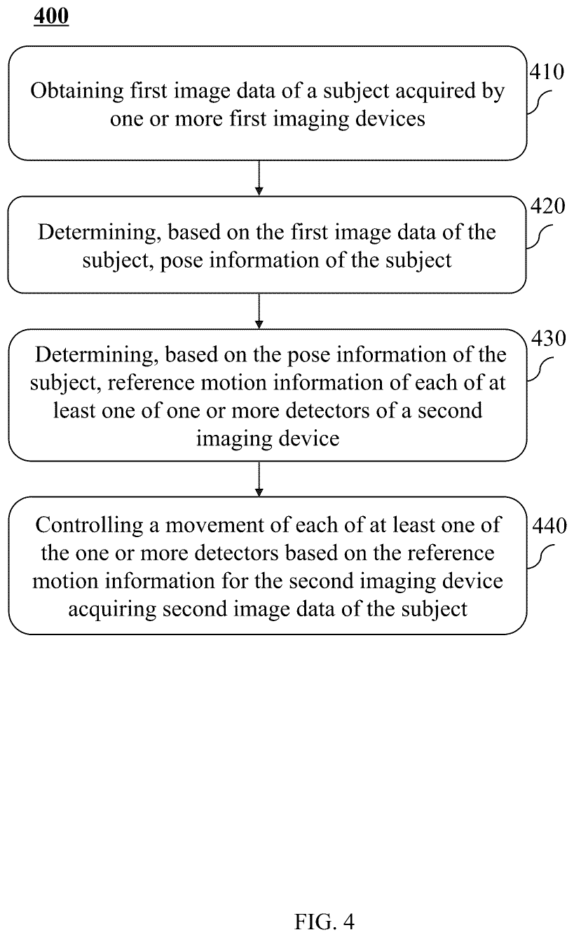

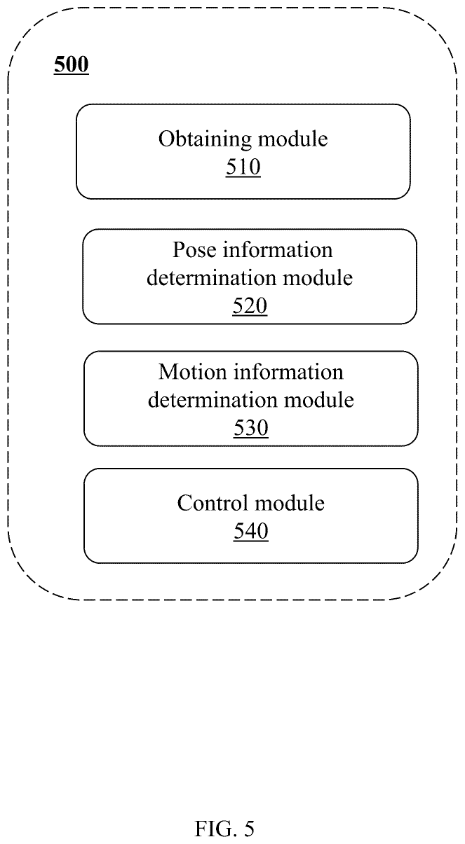

FIG. 4 is a flowchart of an exemplary process for a motion control of a detector according to some embodiments of the present disclosure. As shown in FIG. 4, process 400 includes operations 410-440. Process 400 may be implemented on the imaging system 100 illustrated in FIG. 1. For example, the process 400 may be stored in the storage device 130 and/or the storage device 220 in the form of instructions (e.g., an application), and invoked and/or executed by the processing device 120 (e.g., or one or more modules in the processing device 500 illustrated in FIG. 5). The operations of the illustrated process presented below are intended to be illustrative. In some embodiments, the process 400 may be accomplished with one or more additional operations not described, and/or without one or more of the operations discussed. Additionally, the order in which the operations of the process 400 as illustrated in FIG. 4 and described below is not intended to be limiting.

In 410, first image data of a subject acquired by one or more first imaging devices may be obtained. In some embodiments, operation 410 may be performed by the obtaining module 510.

A first imaging device is an imaging device that acquires a surface contour and/or position information of the subject through non-radioactive means.

In some embodiments, the one or more first imaging devices may include at least one of a three-dimensional camera or a radar device. The three-dimensional camera may include a binocular camera, a structured light camera, or a direct Time-of-Flight (dToF) camera. The radar device includes, but is not limited to, a millimeter-wave radar, a LiDAR, an ultrasonic radar, etc. In some embodiments, the first imaging devices may include optical or depth sensing devices.

More descriptions of the first imaging devices may be found elsewhere in the present disclosure.

In some embodiments, one of the one or more first imaging devices may be arranged on one of the one or more detectors or on a ceiling of a scanning room where the second imaging device is located.

The subject refers to a human object, animal object, or a model (e.g., a phantom) that needs medical scanning. During the scan, the subject may be located on the couch, and the posture of the subject may be dynamic and change before and during the scan, so the posture of the subject needs to be monitored in real time or periodically.

The first image data refers to raw data acquired by at least one of the one or more first imaging devices for characterizing a three-dimensional spatial morphology and position of the subject. The first image data may include but are not limited to point cloud data, a depth image, a three-dimensional contour model, etc., of the subject.

In some embodiments, one of the one or more first imaging devices (such as a 3D camera) may be arranged at a position in the scanning room such that a field of view (FOV) of the first imaging device may cover the subject. For example, the first imaging device may be fixedly installed on the ceiling of the scanning room to acquire the first image data of the subject. The first image data of the subject acquired by the first imaging device installed on the ceiling of the scanning room may represent the whole body of the subject.

In some embodiments, at least one of the one or more first imaging devices (such as a 3D camera) may be arranged at a position in the scanning room such that a field of view of each of at least one of the one or more first imaging devices may cover a portion of the subject. For example, a first imaging device (such as a lidar) may be installed on a detector of the second imaging device. The detector may be controlled to move along an axis of the couch, such that the first imaging device arranged on the detector may move along the axis of the couch to different locations. During the movement of the first imaging device, at each of the different locations, the first imaging device may cover a portion of the subject and acquire first sub-image data of one of different portions of the subject. The first sub-image data of the different portions may be spliced into complete first image data representing the whole body of the subject.

In some embodiments, the second imaging device may include multiple detectors and the multiple detectors may be arranged along the axis of the second imaging device. Each of the first imaging devices may be installed on one of the multiple detectors. The first imaging devices may have different FOVs. FOVs of two adjacent first imaging devices may have an overlapping region. When the subject is located on the couch, each of the first imaging devices may acquire the first sub-image data of a portion of the subject and the first sub-image data of the different portions of the subject may be combined to form the first image data of the subject.

The first image data of the subject may be acquired by the one or more first imaging devices during a scan of the subject or before the scan of the subject.

In 420, based on the first image data of the subject, pose information of the subject may be determined. In some embodiments, operation 420 may be performed by the pose information determination module 520.

The pose information refers to a digital description of the position, an orientation, a surface contour, and/or overall posture of the subject in three-dimensional space. For example, the pose information of the subject may include the positions and angle information of different portions of the subject in a three-dimensional space. As another example, the pose information of the subject may include position information of the surface contour of the subject in the 3D space. The position information of the surface contour may include positions of points on the surface contour (or different portions of the surface contour) of the subject in the 3D space.

In some embodiments, the pose information may be in the form of a three-dimensional reconstruction model of the subject, etc. In other words, the pose information may include a position relationship of different portions of the subject in the 3D space and/or positions of the different portions of the subject in the 3D space.

In some embodiments, the processor may generate the pose information of the subject (such as a three-dimensional reconstruction model) based on the first image data of the subject (such as point cloud data acquired by a LiDAR) through a surface reconstruction algorithm. The surface reconstruction algorithm may include a Poisson reconstruction algorithm, a marching cubes algorithm, etc. The three-dimensional reconstruction model may specifically be a geometric representation of the subject in three-dimensional space, which contains accurate pose information. Taking the LiDAR as an example, before the scan of the second imaging device starts or during the scan of the second imaging device, the LiDAR may acquire the first image data of the subject, and input the first image data into the processing device 120 for processing. The processing device 120 may perform data preprocessing such as denoising, filtering, registration, etc., on the original point cloud data acquired by the LiDAR to improve the accuracy and reliability of the first image data, then use the preprocessed point cloud data to construct a three-dimensional reconstruction model of the subject, and perform smoothing, mesh optimization, etc., on the three-dimensional reconstruction model to improve the precision and usability of the three-dimensional reconstruction model, thereby improving the accuracy of the pose information of the subject. The pose information may be used to achieve motion control of the detector of the second imaging device.

In some embodiments, the pose information may also be in the form of a three-dimensional contour, etc. For example, the pose information may include contour information (also referred to as a first contour) of the subject in space. As a further example, the pose information may include position information of points on the contour of the subject.

In some embodiments, the processing device 120 may control a detector of the second imaging device where a first imaging device is installed to move along an axis of the second imaging device to acquire first sub-image data of the subject. The detector may be stopped at target locations, and the first imaging device may acquire the first sub-image data at each of the target locations. The first sub-image data may represent a portion of the subject. The target locations may be determined based on the FOV of the first imaging device. For example, the target locations may be determined such that the FOVs of the first imaging device at the target locations may cover the whole body of the subject along the axis of the second imaging device. The processing device 120 may determine a first sub-contour of a portion of the subject based on the first sub-image data of the subject and determine the first contour based on first sub-contours of different portions of the subject. In some embodiments, two first sub-contours determined based on first sub-image data acquired at two adjacent target locations may overlap such that the two first-sub-contours may be spliced to form the first contour. For related content of the part, refer to the relevant description below.

In 430, based on the pose information of the subject, reference motion information of each of at least one of one or more detectors of a second imaging device may be determined. In some embodiments, operation 430 may be performed by the motion information determination module 530.

The reference motion information is a motion scheme that guides how the detector moves, enabling the detector to efficiently and safely acquire high-quality image data.

In some embodiments, the reference motion information may include a reference motion path and reference path parameters. The reference motion path may be defined by a plurality of path points. The reference path parameters may be configured to describe information of the path points and/or a motion state of the detector at the path points.

The reference path parameters include a reference path point density, reference distribution positions of a plurality of path points, and a reference angle (orientation) of the detector at each path point, a reference dwell time of the detector at a path point, a reference motion speed of the detector at a path point, etc.

The processing device 120 may determine the reference motion information of each of at least one of the one or more detectors of the second imaging device based on the pose information of the subject through a plurality of ways.

In some embodiments, the processing device 120 may obtain initial motion information based on a first portion of one or more scanning parameters and/or the pose information; and determine the reference motion information by optimizing the initial motion information based on a second portion of the one or more scanning parameters.

The one or more scanning parameters may include a scanning range, a scanning time, a scanning data amount, an expected scan radiation dose, an expected scan image quality, a type of radioactive nuclide, an energy peak of the radioactive nuclide, a corresponding window width of the energy peak of the radioactive nuclide, attribute information of the subject (such as age, height, weight, scan part, and health status), a type of detector collimator, a scan purpose (such as for diagnosis, or for developing a treatment plan, etc.).

The scanning time refers to a duration of each detector for acquiring data at each position, which affects the resolution and noise level of a reconstructed image. In some embodiments, the one or more scanning parameters may also include: a scan angle, an angle range of each detector rotating around the subject, ensuring coverage of the required imaging area; a reference scan speed, a rate at which each detector moves, which affects the speed of data acquisition and image quality; an acquisition sensitivity which affects signal reception and image quality; a gain and an offset that are calibration parameters of the detector, used to ensure signal accuracy and consistency; a time synchronization parameter, ensuring that all detectors start and end the scan at the same time point, which is crucial for image registration and reconstruction; a position calibration parameter, using a calibration source to verify and correct the relative positions and angles between detectors, ensuring image consistency.

In some embodiments, the one or more scanning parameters may be input by a user through a user operation interface based on a scan protocol or set by the system by default.

In some embodiments, the one or more scanning parameters may be divided into two parts, used for two different decision stages. The same parameter may belong to the first portion or the second portion.

The first portion of the one or more scanning parameters refers to parameters used for generating the initial motion information. The second portion of the one or more scanning parameters refers to parameters used for optimizing the initial motion information. In some embodiments, the first portion of the one or more scanning parameters and the second portion of the one or more scanning parameters may have at least one same parameter. In some embodiments, the first portion of the one or more scanning parameters and the second portion of the one or more scanning parameters may be completely different. For example, the first portion may include the scanning range, etc., and the second portion may include the scanning time, scan image quality, etc.

The initial motion information refers to a preliminary motion scheme that is not optimized for guiding how the detector moves.

In some embodiments, the processing device 120 may determine the initial motion information based on multiple preset motion information. Each of the multiple preset motion information may correspond to a posture and/or a set of scanning parameters. The processing device 120 may match the pose information of the subject with the posture and/or the set of scanning parameters corresponding to each of the multiple preset motion information to determine the initial motion information.

In some implementations, the processing device 120 may obtain the first contour of the subject based on the pose information, obtaining a second contour of the couch where the subject is located; splice the first contour with the second contour to obtain a third contour, determine the minimum convex hull of the third contour; and determine the initial motion information based on the minimum convex hull (and may combine other parameters of the first portion such as “safe distance” for expansion), as the initial motion information. For related parameters of the part (such as the first contour, the third contour, the minimum convex hull, etc.), refer to the relevant description below. In some embodiments, the first portion of scanning parameters may include a scanning range, and the processing device 120 may determine at least a portion of the first contour of the subject based on the scanning range and combine the at least a portion of the first contour of the subject with the second contour to form the third contour.

In some embodiments, after obtaining the pose information of the subject, the processing device 120 may combine a scan requirement input by the user (i.e., reference values of the first portion of scanning parameters) to plan the initial motion information of the detector. Specifically, the scan requirement may be a scan requirement that constrains the scan process and scan result of the medical scanning of the subject. The scan requirement may specifically include: a scanning range requirement indicating the scanning range, and a scan quality requirement indicating the scanning time and scanning data amount, etc. For the scanning range, the user may specify a body part or area of the subject that needs to be scanned, such as the heart, lungs, brain, etc. The part or area specified by the scanning range may be an area corresponding to a specific anatomical structure or may be a specific body area delineated according to clinical diagnostic needs. The scanning time may be the duration for medical scanning of the subject. Based on the scanning time and the size of the scanning range, a requirement on the scan speed or the movement speed of the detector may be determined. At the same time, it is also necessary to consider time overhead, such as motion pauses and pose adjustments of the detector during the scan. The scanning data amount may be the data amount that needs to be acquired for medical scanning of the subject. The scanning data amount may be matched by adjusting the density of path points of the detector, the dwell time of the detector at one or more path points, or the data acquisition rate, etc. For example, the density of path points in an area requiring high resolution may be higher than the density of path points in an area requiring low resolution and appropriately reduce the density of path points in areas with low resolution requirements.

In some embodiments, the initial motion information may be determined based on the scanning range and the pose information. The scanning range may be the body part or area of the subject scan requirement that needs to be scanned. By combining the pose information of the subject and the preset scanning range, planning of the initial motion information of the detector is achieved. Specifically, by combining the pose information and the preset scanning range, a preset path planning algorithm may be used to plan the distribution positions of path points in the initial motion information of the detector. The path planning algorithm may be a heuristic search algorithm (A-star algorithm), a Rapidly-exploring Random Trees (RRT) algorithm, or Dijkstra's algorithm that uses a greedy strategy to gradually build a shortest path tree, etc. The path planning algorithm may find an optimal or feasible path from a start point to an end point in three-dimensional space.

Determining the initial motion information based on the scanning range and the pose information may include matching or mapping the scanning range with the three-dimensional reconstruction model of the subject to convert the scanning range into a coordinate range or volume in a three-dimensional space, and then planning the distribution positions of path points in the initial motion information based on the coordinate range or volume delineated in three-dimensional space. For example, based on the scanning range of the subject and the pose information, a motion range of the each of at least one of one or more detectors may be determined; and the initial motion information may be determined based on the motion range. The motion range may be the coordinate range or volume of the scanning range in the 3D space. More descriptions for the motion range may be found elsewhere in the present disclosure.

Performing initial planning of the motion path of the detector according to the pose information of the subject and the scanning range input by the user, so that the planned initial motion information covers the entire scanning range. The planning not only considers how to cover the entire region of interest, but also strives to reduce unnecessary motion, thereby reducing the instability of the detector during motion. Therefore, by obtaining the pose information of the subject, intelligently and automatically planning the motion path of the detector can ensure that the subsequent scan of the detector covers the entire region of interest of the subject, while reducing unnecessary motion of the detector, and can adapt to subjects of different body types.

In some embodiments, the processing device 120 may designate the initial motion information as the reference motion information.

In some embodiments, the processing device 120 may determine the reference motion information by optimizing the initial motion information. For example, the processing device 120 may determine one or more optimization objectives based on the second portion of the one or more scanning parameters and determine the reference motion information by optimizing the initial motion information based on the one or more optimization objectives using an optimization model. The reference motion information satisfies the one or more optimization objectives.

An optimization objective refers to a target that the optimization process needs to achieve or approach, used to guide how the initial motion information is optimized.

In some embodiments, the processing device 120 may determine the optimization objectives based on the second portion of the one or more scanning parameters. For example, if the second portion of the one or more scanning parameters includes the scanning time, an optimization objective may be to minimize the scanning time. As another example, if the second portion of the one or more scanning parameters includes an image quality, an optimization objective may be to maximize the image quality or ensure that the scanning data amount is above a preset threshold.

The optimization model refers to a model for optimizing the initial motion information. In some embodiments, the optimization model may be a mathematical model.

In some embodiments, the optimization model is constructed based on an objective function and one or more constraints on at least one of the reference path parameters in reference motion information. The objective function may be constructed based on the one or more optimization objectives and the one or more reference motion parameters. For example, the reference motion parameters may serve as independent variables and the objective function may be configured to adjust values of the initial path parameters in the initial motion information, so that the output value of the objective function is optimized (i.e., satisfies the one or more optimization objectives), thereby determining the reference path parameters which are the adjusted initial path parameters. The processing device 120 may optimize the initial motion information by performing an iterative process based on the objective function, the one or more constraints, and the one or more optimization objectives.

The objective function is a mathematical function that takes path parameters to be optimized in the initial motion information as independent variables (i.e., decision variables), and the function value of the objective function is used to quantitatively evaluate the “goodness” of the reference motion information (e.g., the motion path and/or the path parameters). The optimization process is to adjust or determine path parameters that change the function value in a desired direction (such as minimization or maximization) (i.e., the one or more optimization objectives).

The one or more constraints refer to a limitation range or value on one or more path parameters, which defines a feasible region of the solution, and any feasible reference motion information must satisfy all constraints.

In some embodiments, the processing device 120 may construct the optimization model based on the objective function, the one or more constraints, and decision variables (i.e., the path parameters).

In some embodiments, the processing device 120 may optimize the initial motion information based on the one or more optimization objectives using the optimization model. For example, the processing device 120 may use a path optimization algorithm (such as particle swarm optimization, genetic algorithm, gradient descent manner) to continuously adjust the path parameters until finding an optimal or approximately optimal solution (i.e., the reference path parameters) that satisfies the optimization objectives and conforms to the one or more constraints, as the reference motion information.

In some embodiments, the processing device 120 may evaluate one or more scanning parameters such as the scanning range, the scanning time, and the scanning data amount to determine the priority of each of the scanning parameters and then determine the optimization objectives based on the priority of each of the scanning parameters. For example, the processing device 120 may determine the second portion of the one or more scanning parameters based on the priorities of the one or more scanning parameters. A scanning parameter with higher priorities may be designated as one of the second portions of the one or more scanning parameters. The processing device 120 may determine the optimization objectives based on the second portion of the one or more scanning parameters. For example, the processing device 120 may designate at least one of the second portions of the scanning parameters as a scanning parameter corresponding to the optimization objectives. As a further example, if the scanning time and radiation dose are the first portion of the one or more scanning parameters, the optimization objectives may include minimizing scanning time and reducing subject radiation dose.

In some embodiments, the priorities of the scanning parameters may be determined by the scan requirements (e.g., low radiation dose, high image quality, emergency diagnosis, anatomical range, etc.) and/or personalized information (e.g., the weight, age, gender, medical history, radiation sensitivity) of the subject. For example, if the scan requirement is emergency diagnosis, the scanning time may be set to the higher priority and one or more scanning parameters associated with the image quality may be set to higher priorities; if the scan requirement is image quality, one or more scanning parameters that affects the image quality may be set to the highest priority; if the subject has a high radiation sensitivity, the radiation dose may be set to the highest priority and/or one or more scanning parameters associated with the radiation dose may be set to have higher priorities. In some embodiments, there may be an interplay relationship between the scanning parameters. For example, there may be an interplay relationship between the scanning time and the scanning data amount, a longer scanning time may allow acquiring more scanning data amount. As another example, the higher scan image quality requires a longer scanning time and/or a higher scan radiation dose. As still another example, the scanning range may also directly affect the scanning time and the scanning data amount. Accordingly, the first portion of the one or more scanning parameters may be determined based on the interplay relationship between the scanning parameters.

During the requirement evaluation process, a plurality of optimization objectives may need to be considered, such as minimizing the scanning time, maximizing image quality, reducing subject radiation dose, etc.

In some embodiments, the one or more constraints may be determined based on the determined optimization objectives, the second portion of the one or more scanning parameters, a physiological condition of the subject, and/or physical parameters of the detector of the second imaging device. The constraints may be configured to constrain the reference path parameters, such that the constraints may be satisfied after the reference path parameters are determined. In some embodiments, a constraint may be configured to constrain the value of at least one of the optimization objectives. For example, an optimization objective may include minimizing the scanning time and a constraint may include keeping the scanning time exceeding a time threshold. In some embodiments, a constraint may be configured to constrain a path parameter directly. For example, the constraint associated with a path parameter may be determined based on a physical parameter of the detector of the second imaging device. The physical parameter of the detector of the second imaging device may include the maximum scan speed, the minimum dwell time, the maximum motion range, etc. The constraint associated with the path parameter may include that the motion speed of a detector is less than the maximum scan speed, or the dwell time exceeds the minimum dwell time, and/or the path points of the detector are within the maximum motion range, etc. In some embodiments, a constraint may be configured to constrain a scanning parameter in the second portion directly. For example, the constraint may limit the radiation dose in a reference range or desired range which is suitable for the subject. The reference range or desired range which is suitable for the subject may be determined based on the physiological condition of the subject. As another example, the constraint may limit the scanning time in a reference range or desired range which is suitable for the subject (e.g., a child). The reference range or desired range which is suitable for the subject may be determined based on the physiological condition of the subject.

For example, when the scan part of this scan is the head of the subject, the scanning range may be determined according to the scan part, and the determined scanning range may be mapped into a preset path planning model in the form of mathematical parameters to determine the motion range. The reference path parameters determined after the optimization of the initial motion information may ensure that the motion range of the detector is within the maximum motion range of the detector.

Similarly, the one or more scanning parameters, such as the scanning time, the scanning data amount, the disease type of the subject, and the scan purpose, may also be mapped respectively. Afterwards, the priority and the interplay between the scanning parameters are evaluated according to the scan requirement.

According to the above second portion of the one or more scanning parameters, the objective function of path planning may be formed. For example, an objective function may include minimizing the scanning time (T) as an optimized objective, maintaining the scan image quality (Q) not lower than a certain quality threshold, and limiting the scan radiation dose (D) within a safe numerical range (i.e., the reference range) as a constraint. In some embodiments, the constraints may include limiting the motion state of the detector to satisfy one or more physical limitations of the detector (such as maximum scan speed, minimum dwell time, etc.) and/or one or more scanning parameters to adapt the subject's physiological conditions (such as radiation sensitivity), thus the objective function may be denoted as the following formula (1):

$$min T = f ( x ) , \qquad ( 1 )$$

where x represents path parameters (such as path point density, dwell time, data acquisition rate, etc.).

The constraints may be denoted as the following formulas (2) and (3):

$$g i ( x ) \leq 0 , \qquad ( 2 )$$

$$hj ( x ) = 0 , \qquad ( 3 )$$

where gi(x) represents an inequality constraint (such as a constraint related to the radiation dose), hj(x) represents an equality constraint (such as a constraint related to the image quality), i=1, 2, . . . , m, j=1, 2, . . . , n, i represents an i-th inequality constraint, and j represents a j-th equality constraint.

Decision variables (i.e., the path parameters) may also be denoted by the following formula (4):

$$x = [ {x}_{1} , {x}_{2} , … , {x}_{k} ] , \qquad ( 4 )$$

where xₖ represents a k-th path parameter.

Therefore, through the above setting of the objective function, the constraints, and the decision variables, the construction of the optimization model is achieved. After constructing the optimization model, a selected path optimization algorithm may be used to solve it. Next, solving based on the particle swarm optimization algorithm is taken as an example for description.

First, the particle swarm may be initialized. Specifically, a group of particles may be randomly generated as a candidate solution of the path parameters. A velocity vector may be assigned to each particle, indicating its movement direction in the search space. After initializing the particle swarm, the group of particles may be evaluated. Specifically, the objective function value of each particle (in the above objective function constructed for minimizing the scanning time, the corresponding objective function value may be the scanning time) may be determined. Whether the path parameters satisfy the constraints (such as image quality and radiation dose limitations) may be determined. After evaluating the group of particles, the group of particles may be updated. For example, according to the objective function value and the constraints, the best position of each particle (i.e., personal optimal solution) may be updated; and the best position of the entire particle swarm (i.e., global optimal solution) may be updated.

After updating the group of particles, the particle velocity and position may be adjusted again. For example, according to a velocity update formula and a position update formula in the particle swarm optimization algorithm, the velocity and position of the group of particles may be updated to ensure that the group of particles move within the search space and avoid falling into a local optimal solution.

The above process of evaluating particles may be performed iteratively to update particles and adjust particles until a predetermined number of iterations is reached or a stopping condition is met (such as the change of the objective function value is less than a certain threshold). Finally, the global optimal solution may be generated in the last iteration, which is the optimal path parameters (i.e., the reference path parameters in the reference motion information).

It may be understood that the application of the above particle swarm optimization algorithm is only an example, and those skilled in the art may also update the above path parameters based on other path optimization algorithms according to the needs of actual application scenarios, which is not specifically limited. In some embodiments of the present disclosure.

The reference path parameters may be a reference path point density, a reference dwell time, a reference data acquisition rate, a reference shape of the path, a reference motion acceleration, a reference motion speed, etc. Specifically, through the optimization model, according to the optimization objectives, the data acquisition rate, shape, speed, acceleration, path point density, and path point dwell time may be iteratively optimized together until the corresponding reference motion path satisfies the above optimization objectives and/or the constraints, and finally the optimization results (i.e., the reference path parameters) of all path parameters are output. The scan achieved based on the motion path corresponding to the adjusted path parameters can satisfy the optimization objectives determined based on the scanning parameters.

For example, the reference path point density and the reference dwell time can make the actual scanning time conform to the scanning time in the scanning parameters or a constraint on the scanning time. The reference path point density can make the actual image quality obtained by the scan conform to the constraint on image quality; or the reference dwell time and the reference path point density can make the final actual radiation dose conform to the constraint on the radiation dose or the radiation dose in the scanning parameters.

Specifically, according to the above scanning parameters, each path parameter of the initial motion information is evaluated and adjusted to ensure that the adjusted path parameters match the scanning parameters. A preset optimization model may be used to optimize and adjust the initial motion information. It may also involve optimization and adjustment of path parameters, such as the shape, speed, acceleration, etc., of the initial motion information. During the optimization process, comprehensive effects of a plurality of factors may be considered, such as scan efficiency, image quality, comfort of the subject, etc.

The optimization model may be constructed based on a preset path optimization algorithm and may optimize the motion path according to the scanning parameters (such as scanning time, data amount, etc.) and preset optimization objectives (such as minimizing scanning time, maximizing image quality, etc.). In some of these embodiments, the optimization model may specifically be implemented based on a path optimization algorithm such as a linear programming algorithm, a nonlinear programming algorithm, a genetic algorithm, a particle swarm optimization algorithm, or the like, or a combination thereof. Optionally, in some embodiments, simulated testing or small-scale experimental verification may be performed on the planned motion path (i.e., the reference motion information) before actual scanning to evaluate the rationality, feasibility, and safety of the motion path.

Furthermore, in some embodiments, according to the optimization objectives, adjusting the path parameters of the path points in the initial motion information so that the reference motion path corresponding to the adjusted path parameters satisfies the optimization objectives may include: determining minimizing the scanning time and the scanning data amount being higher than a preset data amount threshold as optimization, using the optimization model based on the optimization objectives, iteratively adjusting each path parameter in the initial motion information until the reference motion information corresponding to the adjusted path parameters satisfies the optimization objectives.

After different subjects are injected with the same radioactive tracer or imaging agent, the radiation intensity of the radioactive nuclide may differ. When the radiation signal intensity collected during the scan is strong, the optimization model may have a scanning data amount higher than the preset data amount threshold and minimizing the scanning time as the optimization objectives, and iteratively optimize path parameters such as the path point density, the dwell time, the data acquisition rate, the path shape, the motion speed, the motion acceleration, etc., in the initial motion information, thereby obtaining the adjusted reference path parameters. The path point density represents the density of stop points of the detector during the scan. In this way, based on the determined optimization objectives, the optimization model may be used to adjust the path parameters of the initial motion information so that the adjusted reference path parameters satisfy the above optimization objectives of minimizing the scanning time and the scanning data amount being higher than the preset data amount threshold. The above data amount threshold may be determined based on actual application scenarios, for example, in a certain scan scenario, the minimum data amount empirical value sufficient for scan result analysis is taken as the data amount threshold.

Therefore, when it is necessary to optimize the initial motion information so that the optimized motion path may satisfy the optimization objectives of minimizing the scanning time and the scanning data amount being higher than the preset data amount threshold, the optimization model needs to be used to adjust each path parameter of the initial motion information. When the detector performs the scan based on the adjusted motion path, the number of path points stayed will decrease, the time stayed at the path points will also decrease, the data acquisition rate will increase, the speed and acceleration will also increase accordingly, and the shape of the path will be more optimized, thereby reducing the overall scanning time and improving scan efficiency.

Additionally, in some embodiments, according to the optimization objectives, adjusting the path parameters in the initial motion information so that the reference motion information corresponding to the adjusted path parameters satisfies the optimization objectives may specifically include: determining maximizing image quality as the optimization objective, using the optimization model based on the optimization objective, iteratively adjusting each path parameter of the initial motion information until the reference motion information corresponding to the adjusted path parameters satisfies the optimization objective.

Specifically, maximizing image quality may be taken as the optimization objective, and the optimization model may be used to optimize path parameters such as the path point density, the dwell time, the data acquisition rate, the path shape, the motion speed, the motion acceleration, etc., of the initial motion information based on the optimization objective, obtain the path parameters output by the optimization model, and accordingly adjust the initial motion information to obtain the reference motion information. The specific process of the optimization model optimizing the path parameters based on the optimization objective may refer to the above embodiments and will not be repeated here.

In some embodiments of the present disclosure, in order to improve the image quality of the scan of the second imaging device, it is necessary to ensure the collected scanning data amount. Therefore, maximizing image quality may be taken as the optimization objective to adjust the path parameters, thereby increasing the scanning data amount and further improving the image quality.

It may be understood that those skilled in the art may also adjust and optimize each path parameter of the initial motion information based on other scanning parameters, so that the optimized motion path can match the scan requirements.

In some embodiments, the optimization model may be a first trained machine learning model, and the processing device 120 may input the initial motion information and the one or more optimization objectives into the first trained machine learning model to generate the reference motion information.

The first trained machine learning model may be a deep neural network, a reinforcement learning model, other supervised learning models, etc. The input of the first trained machine learning model may include the initial motion information and the one or more optimization objectives, and the output may be the reference motion information.

In some embodiments, the first trained machine learning model may be obtained by training an initial first machine learning model based on first training samples. Each of the first training samples may include historical initial motion information and one or more historical optimization objectives, and a training label of the first training sample may be historical actual motion information corresponding to the historical initial motion information.

In some embodiments, each of the first training samples may be inputted into the initial first machine learning model, a first loss function may be constructed based on the training label of the each first training sample and the output result of the initial first machine learning model by processing the each first training sample, the parameters of the initial first machine learning model may be updated based on the first loss function, and in response to determining that a termination condition is satisfied, the initial first trained machine learning model having updating parameters generated in the last iteration may be designated as the first trained machine learning model. The parameters of the initial first machine learning model may be updated according to the gradient descent manner, and the termination condition may be that the first loss function converges, the number of iterations reaches a threshold, etc.

In some embodiments of the present disclosure, using a machine learning model as the optimization model, its ability to process complex patterns can effectively capture and learn complex, nonlinear “optimization strategies”, and it can improve the intelligence and adaptability of optimizing the initial motion information through continuous learning, so as to quickly and accurately generate the reference motion information.

In some embodiments, the processing device 120 may determine a motion range of the each of at least one of one or more detectors based on a scanning range of the subject and the pose information, and determine the reference motion information based on the motion range.

For example, the processing device 120 may match the pose information of the subject with the preset scanning range to determine the motion range; according to the motion range, plan the distribution positions of path points in the reference motion information of the detector.

By matching or mapping with the three-dimensional reconstruction model of the subject, the scanning range is converted into a coordinate range or volume in a specific three-dimensional space, thereby determining the motion range when the detector performs scanning on the subject in the subsequent medical scanning process. Then, based on the motion range, the reference motion information of the detector is planned.

In some embodiments of the present disclosure, by matching the pose information with the scanning range, the motion range of the detector is determined, and then the reference motion information of the detector is planned, which can improve the rationality and accuracy of the reference motion information planning, and thereby improve the efficiency of subsequent path optimization. In some embodiments, the scanning range of the subject may be not the whole body of the subject and the processing device 120 may map the scan range of the subject to the pose information to determine the position of the scanning range in the space and the position of the scanning range in the space may be the motion range of the detector. The motion range may define the motion path of the detector. The processing device 120 may further determine the reference motion information (e.g., the reference path parameters) based on the motion range according to the embodiments as described in the present disclosure.

In some embodiments, the processing device 120 may also match the pose information of the subject with the preset scanning range to determine the motion range; according to the motion range, determine the initial motion information of the detector; and then determine the reference motion information by optimizing the initial motion information based on the second portion of the one or more scanning parameters. The optimization process of the initial motion information may be referred to the relevant description above.

In some embodiments, the processing device 120 may determine the reference motion information using a second trained machine learning model based on the pose information of the subject.

The second trained machine learning model refers to a model used to generate motion information, and the second trained machine learning model may be a deep neural network, a reinforcement learning model, other supervised learning models, etc.

The input of the second trained machine learning model may include the pose information of the subject, and the output may be the reference motion information.

In some embodiments, the second trained machine learning model may be obtained by training an initial second machine learning model based on second training samples. Each of the second training samples may include historical pose information of a historical subject, and a training label of each of at least a portion of the second training samples may be historical actual motion information of a detector for scanning the historical subject having the historical pose information.

In some embodiments, each of the first training samples may be inputted into the initial second machine learning model, a second loss function may be constructed based on the training labels of the each first training samples and the output result of the initial second machine learning model by processing the each second training sample, the parameters of the initial second machine learning model may be updated based on the second loss function, and in response to determining that a termination condition is satisfied, the initial second trained machine learning model having updating parameters generated in the last iteration may be designated as the second trained machine learning model. In response to determining that the termination condition is satisfied, the parameters of the initial second machine learning model may be updated according to the gradient descent manner. The termination condition may be that the second loss function converges, the number of iterations reaches a threshold, etc.

In some embodiments of the present disclosure, through the second trained machine learning model, end-to-end one-click generation from the pose information to the reference motion information is achieved, greatly improving the system response speed, and based on strong generalization ability, the machine learning model can quickly and reliably provide high-quality reference motion information for various pose information.

In some embodiments, the input of the second trained machine learning model may include the pose information of the subject, at least one of medical image data acquired by a third imaging device, physiological status information of the subject, or the one or more scanning parameters of the subject.

The medical image data refers to medical image data acquired by the third imaging device that displays the internal anatomical structure of the subject. For example, the third imaging device may be a computed tomography (CT) device, an X-ray (XR) device, a magnetic resonance imaging (MRI) device, etc.

In some embodiments, before or simultaneously with the SPECT scan, the third imaging device may be used to scan the subject to obtain the medical image data of the subject.

In some embodiments of the present disclosure, by fusing the medical image data, the second trained machine learning model not only considers the external contour (pose information) when planning the path, but also “sees” the internal organs, bones, and lesion locations. This enables the generated reference motion information to make more targeted adjustments to the orientation, angle, and dwell time of the detector, thereby optimizing data acquisition for specific regions of interest, and for scans of specific lesions such as tumors, it can plan a customized detector path that is most beneficial for imaging the lesion.

The physiological status information refers to data reflecting dynamic physiological activities of the subject, such as the heart rate, the respiratory rate, etc., which may cause periodic or aperiodic motion of the body surface.

In some embodiments, the physiological status information of the subject is determined using the first imaging device. For example, the first imaging device may obtain surface data (i.e., pose information) of the subject at different times, and the surface data at different times may be used to determine the surface displacement data of the subject. The surface displacement data of the subject may be associated with the physiological status information of the subject. The physiological status information may include a heart rate, a pulse amplitude, a respiratory rate, a breathing amplitude, etc.

In some embodiments, after obtaining body surface displacement data, the processing device 120 may determine physiological status information based on the body surface displacement data by a signal processing process, a feature extraction process, an algorithm calculation process, a verification and calibration process, etc. In the signal processing process, the body surface displacement data may be preprocessed through filtering and denoising, and then periodic signals related to heartbeat or respiration may be extracted. In the feature extraction process, feature parameters such as frequency and amplitude may be extracted from the preprocessed signals, and these feature parameters may be directly related to heart rate and respiratory rate. In the algorithm calculation process, algorithms such as Fast Fourier Transform (FFT) and wavelet transform may be used to analyze the feature parameters and calculate the heart rate and respiratory rate. Afterward, verification and calibration are performed, comparing the calculated heart rate and respiratory rate with data from standard measurement devices, such as electrocardiogram acquisition devices and respiratory monitors, and calibration is performed if necessary to improve accuracy.

In some embodiments of the present disclosure, the first imaging device (such as lidar) can be used to accurately measure the tiny body surface displacements caused by heartbeat and respiration. By analyzing the body surface displacement data, the subject's heart rate and respiratory rate may be monitored in real time. The monitoring result can be applied in specific applications such as myocardial perfusion imaging to compensate for the influence of the physiological status information of the subject on the scan results, ensuring the accuracy and reliability of the scan result.

In some embodiments of the present disclosure, by inputting the physiological status information, the second trained machine learning model can predict periodic motion during the scan. Therefore, it can generate intelligent, adaptive reference motion information, for example, planning the detector to perform data acquisition during respiratory intervals, or instructing the detector to perform compensatory movements synchronized with the physiological cycle, thereby effectively reducing motion artifacts and improving image quality.

In some embodiments, the pose information of the subject may be denoted by a first contour of the subject, and the processing device may further obtain a second contour of a couch where the subject is located; determine a third contour by combining the first contour and the second contour; and determine the reference motion information based on the third contour.

The first contour may be a three-dimensional contour of the upper surface of the subject when the subject is located on the couch. The upper surface of the subject when the subject is located on the couch refers to a surface of the subject perpendicular to the surface of the couch and away from the couch. For example, when the subject is supine on the couch, the first contour may be a three-dimensional contour of the ventral surface of the subject; when the subject is prone on the couch, the first contour may be a three-dimensional contour of the dorsal surface of the subject.

The processing device 120 may be connected to the first imaging device and perceive the first contour of the subject through the first imaging device.

In some embodiments of the present disclosure, by perceiving the first contour of the subject through the first imaging device, the contour of the subject can be acquired in a non-radioactive manner, reducing the use of radiation dose.

In some embodiments, when the one of the one or more first imaging devices arranged on the one of the one or more detectors, the processor may control the one of the one or more detectors to move along an axis of the second imaging device such that the first imaging device may acquire first sub-image data of the subject at each of different locations along the axis of the second imaging device. The processing device 120 may determine a first sub-contour of a portion of the subject based on the first sub-image data of the subject and determine the first contour based on first sub-contours of different portions of the subject.

In some embodiments, when each of the one or more first imaging devices arranged on one of the one or more detectors and the one or more detectors are located at different positions along the axis of the second imaging device, the processing device 120 may control each of the first imaging devices to acquire first sub-image data of a portion of the subject. The processing device 120 may determine a first sub-contour of a portion of the subject based on the first sub-image data of the subject and determine the first contour based on first sub-contours of different portions of the subject.

In an exemplary embodiment, the above first imaging device may be installed on the ceiling. The first imaging device installed on the ceiling of the scanning room may cover the entire subject to acquire the first contour of the subject.

FIG. 5 is a schematic structural diagram of a processing device according to some embodiments of the present disclosure.

As shown in FIG. 5, the processing device 500 may include an obtaining module 510, a pose information determination module 520, a motion information determination module 530, and a control module 540.

The obtaining module 510 is a functional unit used for obtaining the first image data of the subject acquired by one or more first imaging devices.

The pose information determination module 520 is a functional unit that determines pose information of the subject based on the first image data of the subject.

The motion information determination module 530 is a functional unit that determines the reference motion information of each of at least one of one or more detectors of a second imaging device based on the pose information of the subject.

The control module is a functional unit that controls a movement of each of at least one of the one or more detectors based on the reference motion information for the second imaging device acquiring second image data of the subject.

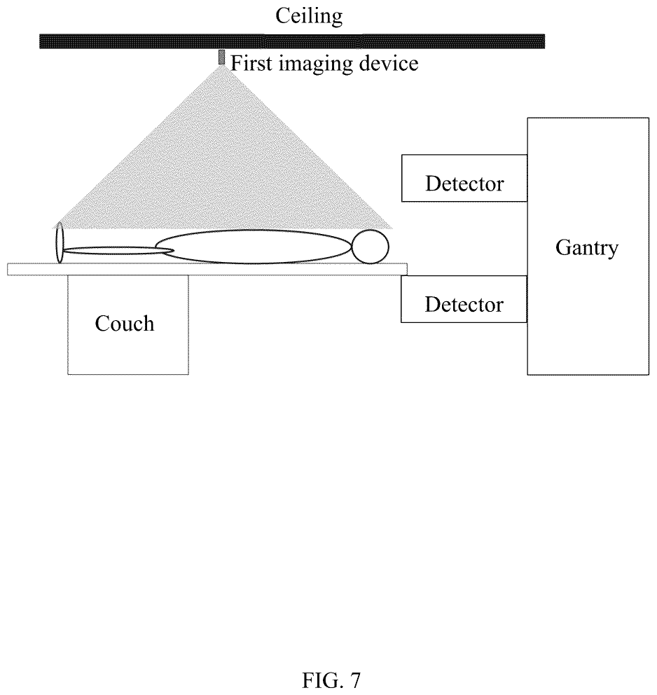

FIG. 7 is a schematic diagram of an installation position of a first imaging device according to some embodiments of the present disclosure.

In specific implementation, referring to FIG. 7, the first imaging device may be installed on the ceiling of the scanning room, specifically on the ceiling within a certain area above the couch. Taking the subject lying supine on the couch as an example, the first imaging device installed on the ceiling may perceive a three-dimensional contour of the ventral surface of the subject and the edge of the couch. The subject and/or the couch may be located within the FOV of the first imaging device installed on the ceiling.

In some embodiments, by installing the first imaging device on the ceiling, the contour of the subject can be acquired in a non-radioactive manner, reducing the use of radiation dose.

It should be noted that the installation position of the first imaging device is not unique. For example, the first imaging device may be installed on a detector of the second imaging device; the step of perceiving the first contour of the subject through the first imaging device may specifically include: controlling the detector of the second imaging device to move in a preset direction (e.g., the axis of the second imaging device), acquiring at least one first sub-contour collected by the first imaging device on the detector at each of different locations in the preset direction; and obtaining the first contour according to the at least one first sub-contour.

The preset direction may be the moving direction of the couch during scanning. The first sub-contour may be a three-dimensional contour of a part of the upper surface of the subject collected by the first imaging device during the movement of the detector.

In specific implementation, the first imaging device may be installed on the detector of the second imaging device, and the processing device may control the detector to move in the moving direction of the couch. The first imaging device may, according to a preset acquisition time interval, sequentially acquire at least one first sub-image data of the subject during the movement of the detector, and process the first sub-image data (such as denoising, segmentation) to extract at least one first sub-contour. The processing device may splice the obtained at least one first sub-contour to obtain the first contour of the subject. The contour splicing manner includes, but is not limited to, point cloud registration or contour matching algorithms, etc. For example, based on overlapping areas between the first sub-contours, a spatial transformation matrix may be calculated to accurately align all first sub-contours to the same coordinate system, so as to splice and form a complete first contour.

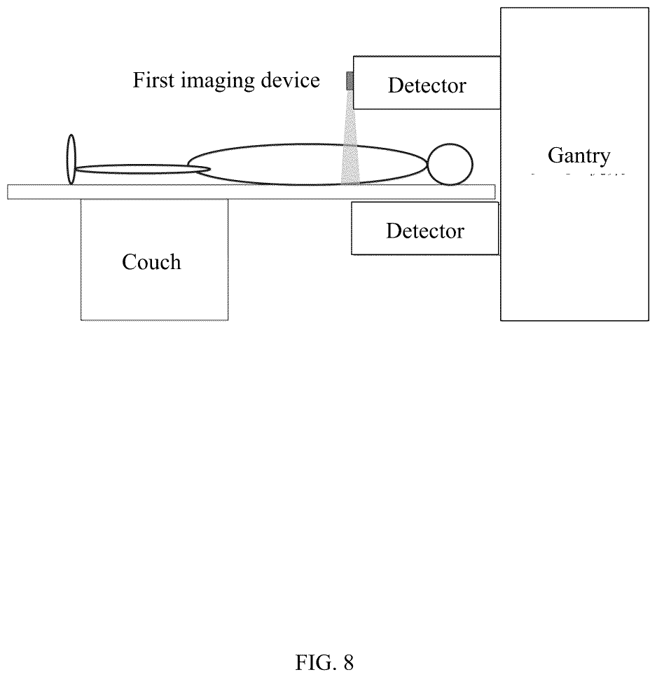

For example, FIG. 8 is a schematic diagram of an installation position of a first imaging device according to some embodiments of the present disclosure. Referring to FIG. 8, the first imaging device may be installed on a detector of the imaging system, and the detector may be controlled to drive the detector to move in the moving direction of the couch. At the first acquisition time, the first imaging device may acquire a three-dimensional contour 1 of the upper surface of a first portion of the subject (e.g., the head); at the second acquisition time, the first imaging device may acquire a three-dimensional contour 2 of the upper surface of a second portion of the subject (e.g., the neck); . . . and so on, until at the n-th acquisition time, a three-dimensional contour n of the upper surface of a n-th portion of the subject (e.g., the feet) is acquired. Then, the three-dimensional contours 1, 2, . . . , n obtained at multiple acquisition times may be spliced to obtain a three-dimensional contour of the entire upper surface of the subject.

It may be understood that, using similar ways, the first imaging device installed on the detector may perceive the edge of the couch.

In some embodiments of the present disclosure, by controlling the detector of the second imaging device to move in a preset direction, acquiring at least one first sub-contour collected by the first imaging device on the detector in the preset direction, and obtaining the first contour according to the at least one first sub-contour, compared with installation on the ceiling, the first imaging device can be closer to the subject, improving the accuracy of the first contour perception, and thereby improving the accuracy of scan planning.

The third contour refers to a combined contour obtained by splicing the first contour and the second contour, representing the outer contour of the subject and the couch as a whole in the scanning space.

In some embodiments of the present disclosure, multi-angle acquisition through the movement of the detector avoids occlusion problems under fixed perspectives and can reconstruct a more complete and accurate whole-body first contour. Moreover, using the existing detector of the system as a mobile platform eliminates the need to install a plurality of fixed first imaging devices in the scanning room, saving costs.

In some embodiments, the processing device may obtain the second contour of the couch through the first imaging device, and according to the relative position relationship between the second contour of the couch and the first contour of the subject, fuse the second contour of the couch and the first contour of the subject to obtain the third contour composed of the upper surface of the subject and the lower surface of the couch.

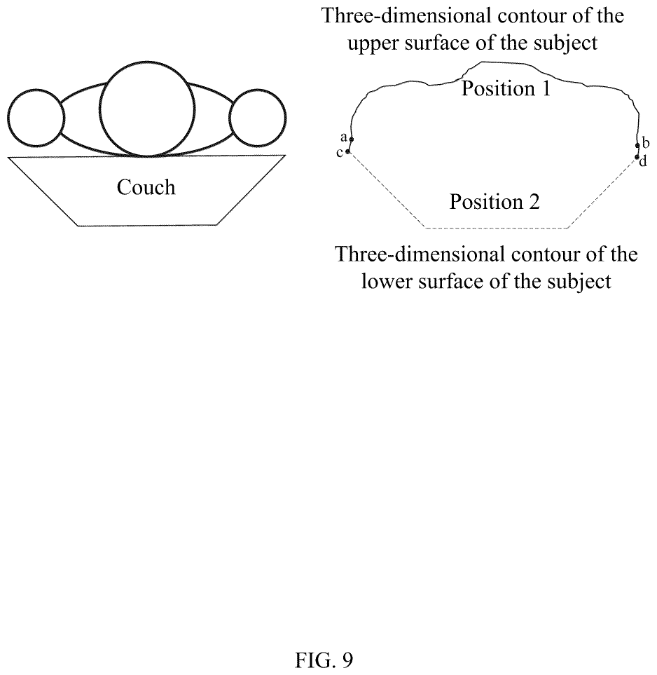

For example, FIG. 9 is a schematic diagram of splicing a top surface contour of a subject and a bottom surface contour of a couch according to some embodiments of the present disclosure. Referring to FIG. 9, the first imaging device may perceive a three-dimensional contour (i.e., the first contour) of the upper surface of the subject while also perceiving the edge of the couch (e.g., the upper edge of the couch as described elsewhere in the present disclosure), thereby obtaining the relative position relationship between the first contour of the upper surface of the subject and the edge of the couch. The processing device 120 may use the edge (e.g., the upper edge) of the couch as a reference, determine position 1 of the first contour of the upper surface of the subject and position 2 of the second contour of the lower surface of the couch according to the relative position relationship, and then fuse the first contour of the upper surface of the subject at position 1 with the first contour of the lower surface of the couch at position 2 to obtain a full contour of the subject and the couch, i.e., the third contour. For example, endpoint a of the first contour of the upper surface of the subject may be connected with endpoint c of the second contour of the lower surface of the couch and endpoint b of the first contour of the upper surface of the subject may be connected with endpoint d of the second contour of the lower surface of the couch to obtain an initial contour, and then smooth the initial contour to obtain the full contour of the subject and the couch, i.e., the third contour.

In some embodiments of the present disclosure, by determining the second contour of the couch, and splicing the second contour with the first contour according to the relative position relationship to obtain the third contour, a full contour of the subject and the couch can be obtained. Based on the full contour to formulate scan planning, the reliability of scan planning can be ensured.

In some embodiments, the second contour may include a first sub-contour and a second sub-contour associated with a surface of the couch where the subject is located, and determining the third contour by combining the first contour and the second contour may include: combining the first sub-contour of the second contour and the first contour via the second sub-contour of the second contour.

The first sub-contour refers to a lower edge contour of the couch. The second sub-contour refers to an upper edge contour of the couch. The upper edge contour of the couch may be the contour of a portion of the surface of the couch where the subject is located excluding a region where the subject is located. The lower edge of the couch may be the contour of a portion of the couch excluding the surface of the couch. According to the upper edge contour of the couch, a position relationship between the first contour and the lower edge of the couch (i.e., the second sub-contour) may be determined. According to the upper edge contour of the couch, a position relationship between the upper edge contour of the couch and the lower edge of the couch (i.e., the second sub-contour) may also be determined. Then the first contour may be combined with the first sub-contour to form the third contour via the second sub-contour.

The first sub-contour may be a default setting of the system. The second sub-contour may be acquired via the first imaging device.

In some embodiments, the processing device 120 may determine a minimum convex hull of the third contour and determine the reference motion information based on the minimum convex hull.

The minimum convex hull refers to the smallest, convex three-dimensional geometric body that completely wraps the third contour (in a two-dimensional schematic diagram, it is a convex polygon).

In some embodiments, the processing device 120 may process the third contour through computational geometry algorithms, for example, using a convex hull algorithm (such as Graham scan, Quick hull algorithm, etc.) to calculate the smallest convex set that may contain all points of the third contour to determine the minimum convex hull.

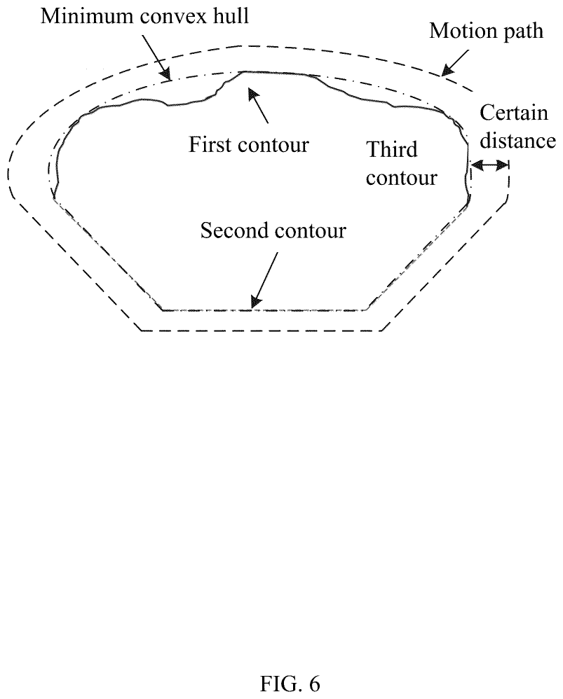

FIG. 6 is a schematic diagram of a contour, a minimum convex hull, and a motion path according to some embodiments of the present disclosure.

In some embodiments, the processing device 120 may, according to a preset safe distance, translate the minimum convex hull outward (away from the third contour) by a certain distance to form an expanded envelope; and use the outer surface of the expanded convex envelope as the motion path or the motion range of the detector, as shown in FIG. 6.

It should be noted that the terminal may also formulate other contents of scan planning according to the expanded convex envelope, including but not limited to scan axial range and detector rotation posture, etc.

In some embodiments of the present disclosure, by expanding the minimum convex hull according to the preset safe distance to obtain the motion path of the detector scan, it can ensure that the detector safely scans the subject, ensuring scan safety.

In some embodiments of the present disclosure, by combining the contour of the couch, the planned reference motion information can more truly reflect the actual physical environment that the detector needs to face in the scanning room, not just the subject itself; it ensures that the generated detector motion path can avoid both the subject and the couch, preventing collision risks and improving the safety of system operation; and the obtained third contour is an ideal input for continuous, smooth scan path planning, laying the foundation for efficient continuous scanning.

In 440, a movement of each of at least one of the one or more detectors may be controlled based on the reference motion information for the second imaging device acquiring second image data of the subject. In some embodiments, operation 440 may be performed by the control module 540.

In some embodiments, a count of the one or more detectors exceeds 1. In other words, the second imaging device may include multiple detectors. The processing device 120 may determine the reference motion information for each of the multiple detectors and control the each of the multiple detectors to move according to the reference motion information.

In some embodiments, the multiple detectors may be arranged along the axis of the second imaging device.

In some embodiments, the processing device 120 may control the one or more detectors to move simultaneously based on the reference motion information of each of the one or more detectors to acquire second image data of different portions of the subject during a same time period.

In some embodiments, the processing device 120 may determine target image data of the subject based on the second image data of the different portions of the subject. For example, the processing device 120 may reconstruct second sub-image of each of the different portions of the subject and splice the second sub-images of the different portions to form the target image data that includes a target image. As another example, the processing device 120 may combine the second image data of different portions of the subject to obtain the target projection data and reconstruct the target image based on the target projection data.

In some embodiments, the imaging system includes a plurality of detectors, and each detector may be used to scan a local area of the subject and obtain corresponding local second image data. For example, the imaging system may be provided with three detectors, and the three detectors may be used to scan and image the head, the chest, and legs of the subject respectively, thereby obtaining head second image data, chest second image data, and leg second image data, respectively.

In some embodiments, the second imaging device may be set to one structure of a single-detector, double-detectors, triple-detectors, or full-ring type as long as it may complete the scanning of the corresponding local area of the subject. The structure of single-detector, double-detectors, triple-detectors, or full-ring type refers to a count of detectors of the second imaging device. For example, the structure of single-detector refers to that there is only one detector of the second imaging device. The structure of double-detectors refers to that there are two detectors of the second imaging device at an axial position. The structure of triple-detectors refers to that there are three detectors of the second imaging device. The structure of full-ring type refers to that the circumferential direction of the second imaging device at an axial position is arranged with multiple detectors and adjacent two detectors have a space less than a threshold (e.g., 0, a length of a detector along the circumferential direction of the second imaging device). During scanning, the second image data may be acquired by each detector at a certain frequency, and the second image data may include timestamps and signal positions indicating which one detector acquire the second image data, and with the second image data acquired by the plurality of detectors may be aligned to a preset coordinate system to uniformly integrate and analyze the second image data obtained from simultaneous scanning, thereby obtaining a plurality of local second image data.

In some embodiments, the second imaging device may include multiple imaging modules, and each of the multiple imaging modules includes at least one of the one or more detectors and a robot joint assembly for providing multiple movement freedoms of the one of the one or more detectors. In some embodiments, each of the multiple imaging modules may include a first controller configured to control a component of the robot joint assembly to move based on the reference motion information. The first controller may be a part of a processor. In some embodiments, each of the multiple imaging modules may include the data processing unit may be a part of the processor.

Specifically, each detector may obtain ray signals based on radiation rays (e.g., y-rays, x-rays, etc.) emitted from the corresponding local area of the subject. In some embodiments, a detector may include a scintillator and at least one of a photodiode or a photomultiplier tube (PMT).

The scintillator may be configured to convert the detected high-energy radiation rays into lower-energy but large-number light signals, and the photomultiplier tube or the photodiode may be used to convert the light signals into electrical signals, thereby obtaining ray signals.

In some embodiments, the detector may also include a photon-counting detector. Unlike the scintillator, the photon-counting detector may be configured to directly convert each incident X-ray or gamma-ray photon into the electrical signals and count them. For example, the photon-counting detector may include a semiconductor sensor and a dedicated readout circuit connected thereto.

The semiconductor sensor may be configured to directly receive radiation photons and generate electron-hole pairs within it; the readout circuit includes multiple independent photon-counting channels, where each channel can be set with an energy threshold to discriminate and count pulse signals from different energy ranges, thereby directly acquiring digital signals with energy information.

In some embodiments, a collimator may be located between the detector and the subject and be used to limit the range and direction of radiation rays entering the detector, only allowing radiation rays within a certain incident direction and range to pass through. It may be understood that the collimators of the plurality of detectors may be different. For example, in the case where the detector has a double-detectors structure, the two detectors may use different collimators, which may be used in daily calibration systems or flexibly selected according to scan requirements in actual use.

In some embodiments, a data processing circuit (e.g., a readout circuit) may be communicatively connected to the at least one detector and may be used to obtain the electrical signals sent by the detector. The data processing circuit may collect, amplify, and shape the electrical signals and convert the electrical signals into the digital signals. For example, the data processing circuit may include a preamplifier, a main amplifier, a pulse shaper, an analog-to-digital converter, or the like, or a combination thereof. In some embodiments, the data processing unit may process the digital signals to generate the second image data of the subject. For example, the data processing circuit may perform an offset correction, a gain correction, a scatter correction, a log transform, or the like, or a combination thereof, on the digital signals to generate the second image data (e.g., second projection data). In some embodiments, each of the plurality of detectors may be connected with a data processing circuit and the data processing circuit may be configured to process the electrical signals from the connected detector. In some embodiments, the plurality of detectors may share a data processing circuit for processing the electrical signals from the plurality of detectors. In some embodiments, the data processing circuit may be integrated into the connected detector.

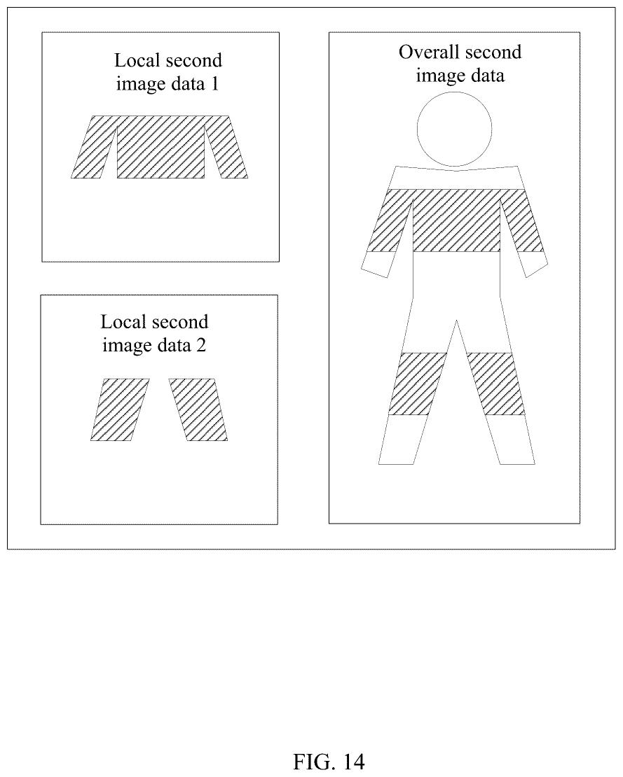

In some embodiments, a data processing unit may be communicatively connected to the data processing circuit and may be used to obtain the digital signals by processing the electrical signals. In some embodiments, the data processing unit may be a part of a processor (e.g., the first controller, the second controller, etc.). In some embodiments, the data processing unit and the data processing circuit may be integrated into one single structure. After receiving the corresponding electrical signals, the data processing unit converts these electrical signals into digital signals (e.g., local second image data) according to a preset data processing algorithm. The local second image data may be tomographic image data or projection image data, and the local second image data can reflect the distribution of radioactive nuclides or anatomical structure inside the local area of the subject, thereby helping doctors in disease diagnosis. After receiving the plurality of local second image data, local second image data generated by multiple detectors may be integrated into the final overall image data. For example, the local second image data may be reconstructed to generate a local image, and multiple local images may be combined to generate a target image. As another example, local second projection data may be combined to form the target projection data of the whole subject, and the target projection data may be reconstructed to form the target image of the subject. During the integration process, the plurality of local second image data may be integrated by using position information in the generated local second image data, so that overlapping parts in the local second image data are integrated together, and the final overall second image data is obtained, as shown in FIG. 13. It may be understood that in the case where the plurality of local second image data do not completely cover all parts of the subject, the generated overall second image data may have blank areas, as shown in FIG. 14. FIG. 13 is a schematic diagram of determining an overall image based on local images of different portions according to some embodiments of the present disclosure. FIG. 14 is a schematic diagram of determining an overall image based on local images of different portions according to other embodiments of the present disclosure.

In some embodiments, when the imaging system or the second imaging device includes multiple detectors, the multiple detectors may be arranged along the axis of the second imaging device. The distribution of detectors in the imaging system of the present disclosure is described in detail below with several specific embodiments.

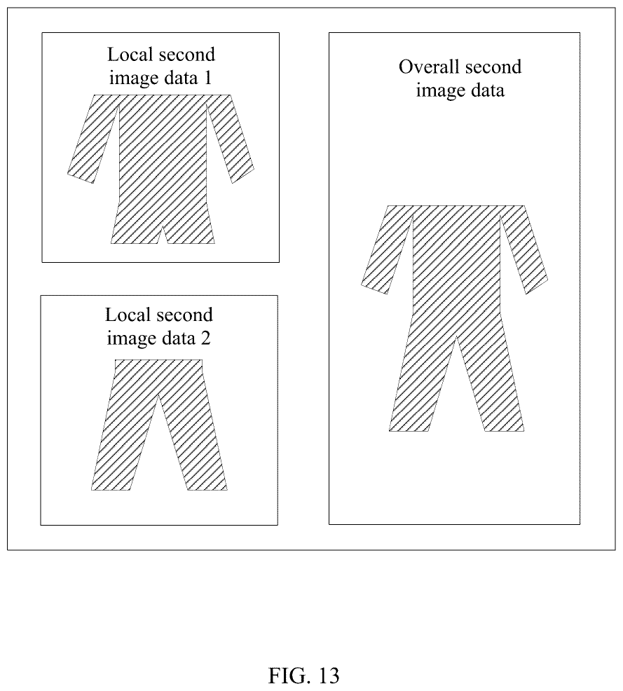

FIG. 10 is a schematic diagram of an exemplary detector distribution in an imaging system according to some embodiments of the present disclosure. FIG. 11 is a schematic diagram of another exemplary detector distribution in an imaging system according to some embodiments of the present disclosure. FIG. 12 is a schematic diagram of another exemplary detector distribution in an imaging system according to some embodiments of the present disclosure. As shown in FIG. 10, the imaging system is provided with three imaging modules, namely an imaging module 1, an imaging module 2, and an imaging module 3, which are used for scanning and imaging the leg, the chest, and the head respectively. Each imaging module is set to a single-detector structure; that is, the imaging module 1 consists of a detector 1, the imaging module 2 consists of a detector 2, and the imaging module 3 consists of a detector 3.

As shown in FIG. 11, the imaging system is provided with two imaging modules, namely an imaging module 1 and an imaging module 2, which are used for scanning and imaging the leg and the head, respectively. Each imaging module is set to a double-detector structure, that is, the imaging module 1 consists of a detector 1 and a detector 2, and the imaging module 2 consists of a detector 3 and a detector 4.

As shown in FIG. 12, the imaging system is provided with three imaging modules, namely an imaging module 1, an imaging module 2, and an imaging module 3, which are used for scanning and imaging the leg, the chest, and the head respectively. Each of the imaging module 1 and the imaging module 2 are set to a single-detector structure, and the imaging module 3 is set to a double-detector structure; that is, the imaging module 1 consists of a detector 1, the imaging module 2 consists of a detector 2, and the imaging module 3 consists of a detector 3 and a detector 4.

In the imaging system of the embodiment, since different imaging modules including a plurality of detectors may scan and image different local areas, and scanning processes of the detectors are relatively independent, when a plurality of local areas need to be scanned and imaged, a plurality of detectors may perform scanning and imaging simultaneously, thereby performing parallel scanning on the subject, improving scanning efficiency. Moreover, when the plurality of detectors work simultaneously, the plurality of detectors can simultaneously capture radiation rays from different local areas, enhancing the flexibility and adaptability of the system. The plurality of detectors working in parallel can collect data more comprehensively and evenly, thereby generating images with higher resolution, greater clarity, and less noise, reducing data loss and artifacts caused by subject movement or repositioning. At the same time, the scanning time can be shortened to reduce subject discomfort and improve the subject's examination experience. Furthermore, due to the shortened examination time, subjects can complete the examination and leave the hospital in a shorter time, reducing their stay time in the hospital. The plurality of detectors enable the imaging system to adapt to various examination needs, whether for a single symptom or a plurality of suspected diseases, it can be completed quickly and accurately.

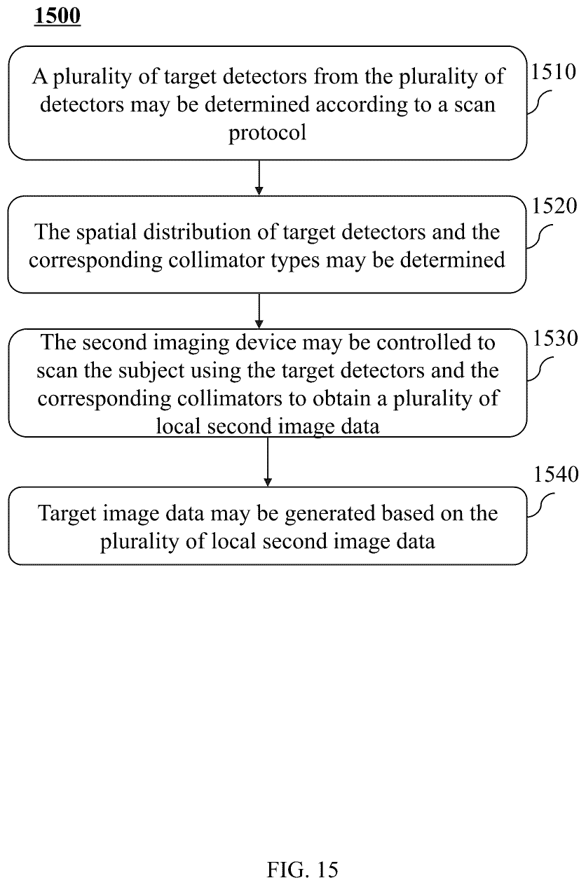

The first controller controls the one or more detectors to move simultaneously based on the reference motion information of each of the one or more detectors to acquire second image data of different portions of the subject during a same time period, and the data processing unit determines target image data of the subject based on the second image data of the different portions. A specific embodiment may include the following steps as shown in FIG. 15:

FIG. 15 is a schematic flowchart of an imaging manner of an imaging system according to some embodiments of the present disclosure.

In 1510, a plurality of target detectors from the plurality of detectors may be determined according to a scan protocol.

Specifically, the user determines the body parts that need to be detected based on the symptoms of the subject and evaluates whether multi-detector parallel imaging is required. Upon determining that multi-detector parallel imaging is required, the user inputs corresponding control commands to the imaging system through a user operation interface. The imaging system may determine the corresponding scan protocol based on the input control commands and select the plurality of target detectors according to the scan protocol. The target detectors are detectors that need to perform scanning and imaging, and each target detector has at least one corresponding first controller. The first controller is responsible for controlling the motion path of the detector to ensure that the detector may accurately align with specific local areas of the subject.

In 1520, the spatial distribution of the target detectors and the corresponding collimator types may be determined.

Specifically, after the imaging system determines the target detectors, it then determines the spatial distribution of the plurality of target detectors. The spatial distribution is used to describe the relative positions between the plurality of target detectors. At the same time, for each target detector, it is also necessary to determine the collimator type corresponding to the target detector. The collimator type is used to determine the field of view and resolution of the detector, and the collimator type may be determined according to the specific type of scanning and imaging required.

In 1530, the second imaging device may be controlled to scan the subject using the target detectors and the corresponding collimators to obtain a plurality of local second image data.

Specifically, after the imaging system determines the target detectors, it uses the target detectors and the corresponding collimators to scan the local areas of the subject, thereby obtaining the corresponding plurality of local second image data.

It may be understood that during the parallel scanning process using the plurality of target detectors, a scanning parameter may be set for each target detector to adapt to the scanning requirements of different parts. The imaging system will determine the corresponding scanning parameters according to different parts (such as head, chest, abdomen, etc.).

In 1540, target image data may be generated based on the plurality of local second image data.

Specifically, after scanning the local areas of the subject and obtaining a plurality of local second image data, the imaging system integrates the local second image data according to relevant parameters of the local second image data, thereby obtaining the overall target image data.

In some embodiments, the target image data may include a target image of the subject and the local second image data may include local second projection data. The processing device may generate the target image by reconstructing local second images based on the local second projection data and combining the local second images to generate the target image. In some embodiments, the processing device may combine the local second projection data to generate target projection data and reconstruct the target image based on the target projection data.

In some embodiments, the process of determining the target image data includes: image preprocessing, preprocessing the local second image data captured by each detector, including denoising, enhancing contrast, etc., to improve image quality; feature extraction: extracting feature points or feature regions from the preprocessed images, these feature points or regions should have similar performance in different images and are used for subsequent registration; image registration: using the extracted feature points or regions, aligning the images captured by different target detectors through registration algorithms (such as affine transformation, rigid transformation, or more complex nonlinear transformations) to ensure that the same anatomical structure in the images is located at the same position in different images; image fusion: after registration is completed, fusing the images from different detectors to generate a complete target image data. During the fusion process, differences such as brightness and contrast between images need to be considered to ensure the quality of the fused image; quality assessment: assessing the quality of the fused image, checking for obvious stitching seams, artifacts, etc. If necessary, the stitching parameters may be adjusted and stitching may be performed again.

In some embodiments, local second images acquired by target detectors with different collimators may also be integrated, but the specific process will be somewhat different from the integration of images acquired by the same collimator. The following is the process of integrating images acquired by target detectors with different collimators to obtain target image data: collimator correction: before stitching and integration, collimator correction needs to be performed on images acquired by different collimators. This is because different collimators will cause differences in image resolution, contrast, etc. Through correction, these differences can be minimized, allowing images acquired by different collimators to have better consistency during stitching. The process of integrating images acquired by target detectors with different collimators may also include image preprocessing and feature extraction, same as stitching images from the same collimator. But due to the differences in collimators, the feature extraction process is more refined and complex. The process of integrating images acquired by target detectors with different collimators may also include image registration. During the registration process, the impact of different collimators on the images needs to be considered, and more complex registration algorithms or additional registration parameters need to be adopted to ensure accurate alignment of the images. The process of integrating images acquired by target detectors with different collimators may also include image fusion and correction. During the fusion process, in addition to considering differences such as brightness and contrast between images, the impact of collimator differences on the fusion result also needs to be considered. The fusion algorithm needs to be adjusted to achieve better fusion results. At the same time, further correction is needed after fusion to eliminate possible stitching seams and artifacts; quality assessment and adjustment: assess the quality of the fused image, check for obvious stitching problems, and if necessary, adjust the stitching parameters and correction algorithm, and re-perform stitching and correction.

In some embodiments, during the process of controlling the detector to move relative to the subject, the system is configured to collect body surface displacement data of the subject; after the single-photon emission computed tomography system completes the scanning of the subject, correcting the above local second image data based on the body surface displacement data.

During the scanning process of the SPECT system, lidar may be used to continuously monitor the tiny body surface displacement data of the subject caused by heartbeat and respiration. Afterwards, physiological status information such as heart rate and respiratory rate is obtained based on the body surface displacement data. Finally, the detection data is corrected using the physiological status information, or the physiological status information is output together with the detection data for detection data analysis. Based on the physiological status information, data post-processing techniques, such as using image reconstruction algorithms or motion correction software, may be used to correct the detection data, improving the accuracy of medical scanning.

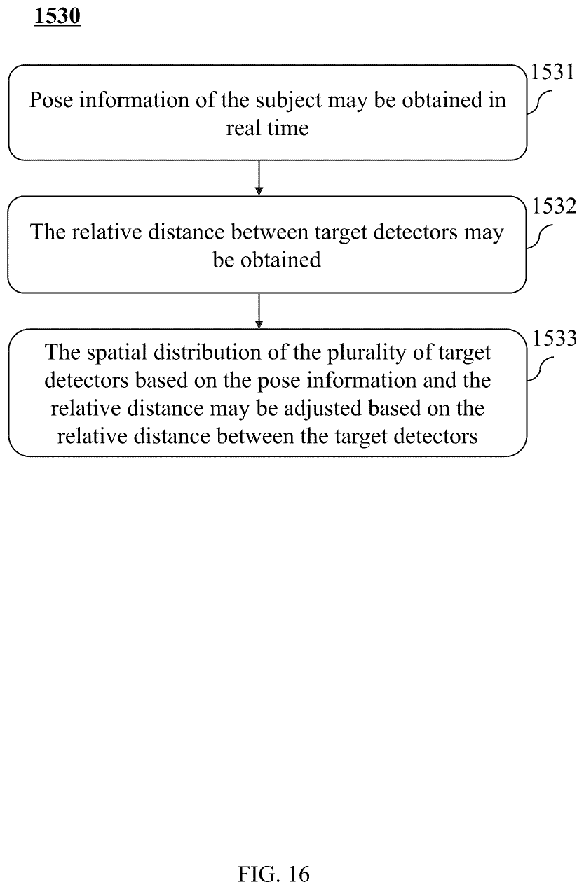

FIG. 16 is a schematic flowchart of scanning the subject according to some embodiments of the present disclosure. As shown in FIG. 16, in the above operation 1530, the step of scanning the subject using the target detectors and the corresponding collimators includes:

In 1531, pose information of the subject may be obtained in real time.

Specifically, the imaging system of the embodiment also acquires the pose information of the subject in real time through the first imaging device during the process of scanning the subject. The posture changes of the subject may be monitored in real time through the second imaging device, such as infrared sensors, cameras, lidar, etc. After the pose information is obtained, the pose information to a central motion control unit as described elsewhere in the present disclosure. After receiving the pose information, the central motion control unit dynamically adjusts the motion control information based on the pose information, so that the motion path of the detector better matches the current posture of the subject, improving the quality of the obtained local second image data.

In particular, in the embodiment, during the process of controlling the detector to move relative to the subject according to the reference motion path, the above motion control method may further include: performing posture detection on the subject in real time, and upon determining that the current pose information of the subject has changed, generating a new reference motion path for the detector based on the current pose information of the subject combined with physiological status information of the subject, and controlling the detector to move relative to the subject according to the new reference motion path.

The embodiment can acquire immediate pose information of the subject during the scanning process and respond immediately to special situations, such as movement of the subject, accurately adjusting the motion path of the detector, thereby improving the accuracy and flexibility of the detector scanning.

In 1532, the relative distance between target detectors may be obtained.

Specifically, the imaging system also acquires the relative distance between each target detector in real time during the process of scanning the subject. When determining the relative distance between each target detector, a fixed point on the gantry is taken as the origin to establish a Cartesian coordinate system. It is ensured that the origin is stable and easily identifiable in space. The position of each target detector may be mapped into these three-dimensional coordinates to determine the relative distance between the target detectors. Precise ranging instruments (such as laser rangefinders) or integrated positioning systems (such as indoor GPS, UWB positioning systems, etc.) may be used to determine the initial positions of the gantry, detectors, subject bed, and the subject, and map these positions into the three-dimensional coordinate system. After the positions in the coordinate system are determined, a mathematical model may be constructed to calculate the relative distance between the detectors to ensure that the detectors do not interfere with each other during movement.

In 1533, the spatial distribution of the plurality of target detectors based on the pose information and the relative distance may be adjusted based on the relative distance between the target detectors.

Specifically, traditional detector motion control manners usually move based on preset scanning paths and cannot respond to subject posture changes in real time. The imaging system in the embodiment of the present disclosure adjusts the spatial distribution of a plurality of target detectors based on the pose information of the subject and the relative distance between each detector during the process of scanning the subject. By dynamically adjusting the detector path, the system can avoid unnecessary repeated scans, thereby reducing subject radiation exposure. At the same time, since the detectors can more flexibly adapt to subject posture changes, radioisotope-labeled drugs can be used more effectively, improving scanning efficiency.

In some embodiments, the second imaging device provided by the imaging system may include multiple detectors. The multiple detectors may consist of multiple imaging modules. Each of the multiple detectors may include at least one detector.

In some embodiments, at least a portion of the detectors may be arranged along an axis of the second imaging device.

It may be understood that the detectors in the embodiments of the present disclosure are generally arranged along the axis of the second imaging device (such as the length direction of the bed board of the couch) to scan and image different local areas of the subject, respectively.

In some embodiments, each of the multiple imaging modules may include at least one of the detectors and a robot joint assembly for providing multiple movement freedoms of the at least one of the detectors.

The robot joint assembly includes one or more robot joints, one or more robotic arms, a driver, etc.

Each detector may be connected to one of the one or more robot joints. The robot joint may have multiple movement freedoms, which enable the detector to perform complex movements in three-dimensional space. This flexibility allows the detector to cover a larger area to adapt to subjects of different body sizes. The robot joint may include a rotational joint, a translational joint, etc. The robotic arm may be configured to control the detector to move according to a predetermined path (e.g., the reference motion information) through joints (such as rotational joints, translational joints, etc.) and the driver (such as a motor, a hydraulic cylinder, etc.), so that the movement of the detector may reach multiple movement freedoms (e.g., 6 degrees of freedom, namely 3 translational degrees of freedom and 3 rotational degrees of freedom).

During scanning, the detector performs operations such as rotation through the robot joint to achieve the required angle orientation, the scanning range, etc.

In some embodiments, each of the multiple imaging modules may include a first controller, configured to control the robot joint assembly to move based on the reference motion information.

The first controller may control the one or more robot joints to move based on the reference motion information, which can achieve complex motion paths of the detector. The controller may include a processor (e.g., a microprocessor, CPU). In some embodiments, the first controller may include a motion control unit. The motion control unit may be a portion of the first controller for motion control of the detector. By controlling the rotation and/or translation of the one or more robot joints through motion programs set in the motion control unit, complex motion paths of the detector can be achieved. In some embodiments, the first controller may include a data processing unit. The data processing unit may be a portion of the first controller for processing data (e.g., the electrical signals and/or the digital signals) acquired by the detector.

In some embodiments, the robotic arm may be equipped with various sensors to detect parameters such as the position, angle, and/or speed of the detector. These sensors feedback real-time data (also referred to as sensing data) to the motion control unit, which then makes necessary adjustments and optimizations based on the sensing data to ensure that the motion path of the detectors meets the preset requirements (e.g., the reference motion information). In this way, the robotic arm can precisely control the motion path of the detector, ensuring that the detector collects data at the optimal position and angle, thereby improving the clarity and resolution of the obtained local second image data.

In some embodiments, at least one of the multiple imaging modules may be detachably connected with the second imaging device.

In some embodiments, at least one of the multiple detectors may be detachably connected with the second imaging device.

In some embodiments, at least one of the multiple imaging modules may be detachably set on the gantry through corresponding robotic arms. The robotic arms may independently control the corresponding detector to move.

Specifically, the gantry is the supporting structure of the entire imaging device, ensuring that all components operate stably and accurately. The detector may be the key component for capturing radiation signals, responsible for converting the received radiation signals into data usable for image reconstruction. The robotic arms may allow the detector to be flexibly installed on the gantry, while also facilitating disassembly and maintenance. This setup enables the imaging device to be quickly adjusted according to different examination needs, improving equipment utilization and adaptability. With the continuous development of medical imaging technology, the performance and functionality of detectors are also constantly improving. By connecting detectors through robotic arms, users can easily replace old detectors and upgrade them to new, more efficient detectors. The independent control capability of the robotic arms means that each detector can be moved to the optimal position as needed to capture the clearest images, helping to reduce examination time and improve diagnostic accuracy. It also allows users to perform personalized settings and adjustments based on the specific situation of the subject and examination requirements.

In some embodiments, the second imaging device may further include a second controller. The second controller may be in communication with the first controller of each of the multiple imaging modules. The second controller may be configured to determine the reference motion information of each of the one or more detectors based on the pose information, the one or more scanning parameters, and position information of the detectors, and transmit the reference motion information of each of the one or more detectors to a corresponding first controller. In some embodiments, the second controller may obtain the position information of the detectors during the movement of the detectors and update the reference motion information based on the position information of the detectors to avoid collisions between detectors.

In some embodiments, the second controller may include a motion control unit (also referred to as a central motion control unit). The central motion control unit may be a portion of the second controller for motion control of the detectors.

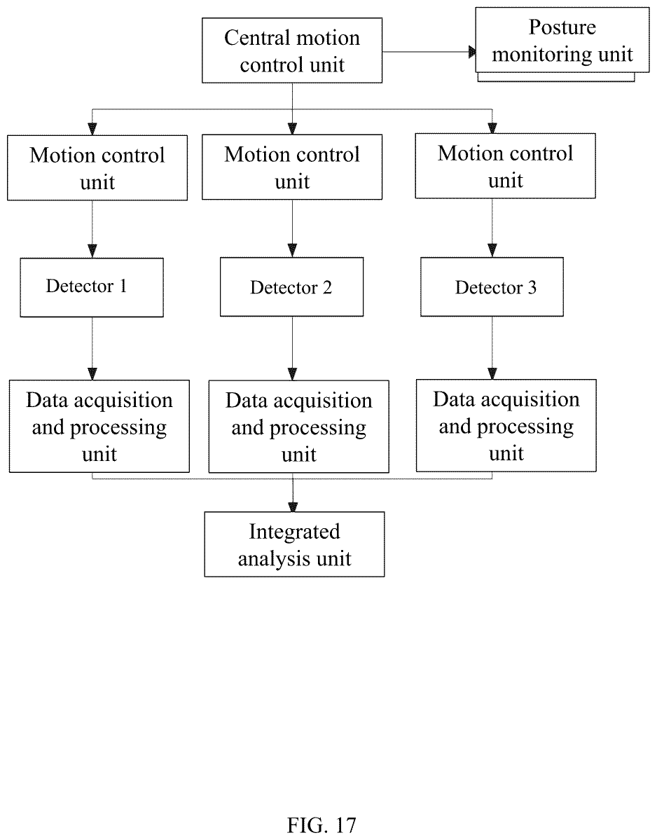

For example, FIG. 17 is a schematic diagram of a second imaging device according to some embodiments of the present disclosure.

As shown in FIG. 17, each imaging module may include a detector and an independent motion control unit, as well as a data processing unit. The motion control unit and/or the data processing unit may be in communication with a central motion control unit to achieve coordinated motion and unified data processing. The gantry in the present disclosure may form a scalable integrated unit with the detectors, allowing configuration according to the actual needs of the system. Each detector may be connected via a robot joint assembly (not shown in FIG. 17), and the gantry may utilize one or more robot joints (such as rotational joints, translational joints, etc.) and a driver (such as a motor, a hydraulic cylinder, etc.) to control the detector to follow a specified motion path. The central motion control unit may be provided to coordinate all the detector motion control units, thereby achieving synchronized control of all detectors while avoiding motion interference between them. For the synchronized control of the detectors, a spatial three-dimensional coordinate system may be established using the couch or gantry as a reference. Based on the coordinate system, the relative positional relationships between the detectors may be determined, allowing for coordinated control without mutual interference. Furthermore, monitoring units such as distance sensors can be incorporated to enhance the safety and robustness of motion control. The central motion control unit may also be coupled with a posture monitoring unit to process real-time data from the posture monitoring unit (e.g., the first imaging device) that reflect the subject's posture, enabling dynamic adjustment of the detector motion paths.

Moreover, to distinguish signals detected simultaneously by the detectors, the imaging system may perform separate data acquisition and processing for each detector to generate the second image data of different portions of the subject. The second image data of different portions of the subject may be processed by the integrated analysis unit to generate the target image of the subject.

Additionally, the way enables unified integrated analysis of simultaneously scanned signals by employing timestamps recorded at a specific frequency in the real-time data, along with recorded signal positions, in conjunction with the three-dimensional coordinate system.

The basic concepts have been described above, and it is apparent to those skilled in the art that the foregoing detailed disclosure serves only as an example and does not constitute a limitation of the present disclosure. While not expressly stated herein, a person skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the disclosure uses specific words to describe embodiments of the disclosure. Such as “an embodiment,” “an embodiment,” and/or “some embodiment” means a feature, structure, or characteristic associated with at least the embodiment of the present disclosure. Accordingly, it should be emphasized and noted that two or more references in the present disclosure, at different locations, to “the embodiment,” or “an embodiment,” or “an alternative embodiment” in different places in the present disclosure do not necessarily refer to the same embodiment. In addition, certain features, structures, or characteristics of one or more embodiments of the present disclosure may be suitably combined.

Furthermore, unless expressly stated in the claims, the order of the processing elements and sequences, the use of numerical letters, or the use of other names as described in the present disclosure are not intended to qualify the order of the processes and methods of the present disclosure. While some embodiments of the present disclosure that are currently considered useful are discussed in the foregoing disclosure by way of various examples, it should be appreciated that such details serve only illustrative purposes, and that additional claims are not limited to the disclosed embodiments. Rather, the claims are intended to cover all amendments and equivalent combinations that are consistent with the substance and scope of the embodiments of the present disclosure. For example, although the implementation of various components described above may be embodied in a hardware device, it may also be implemented as a software-only solution, e.g., an installation on an existing server or mobile device.

Similarly, it should be noted that in order to simplify the presentation of the disclosure of the present disclosure, and thereby aid in the understanding of one or more embodiments of the present disclosure, the foregoing descriptions of embodiments of the disclosure sometimes group a plurality of features together in a single embodiment, accompanying drawings, or in a description thereof. However, the method of disclosure does not imply that more features are required for the objects of the present disclosure than are mentioned in the claims. Rather, claimed subject matter may lie in less than all features of a single foregoing disclosed embodiment.

Some embodiments use numbers to describe the number of components, attributes, and it should be understood that such numbers used in the description of an embodiment are modified in some examples by the modifiers “about,” “approximately,” or “substantially,” “approximately,” or “generally” is used in some examples. Unless otherwise noted, the terms “about,” “approximate,” or “approximately” indicate that a ±20% variation in the stated number is allowed. Correspondingly, in some embodiments, the numerical parameters used in the disclosure and claims are approximations, which may change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments, such values are set to be as precise as practicable.

For each of the patents, patent applications, patent application disclosures, and other materials cited in the present disclosure, such as articles, books, disclosure sheets, publications, documents, and the like, are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions, and/or use of terms in the present disclosure shall control. use shall prevail.

Finally, it should be understood that the embodiments described in the present disclosure are only used to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A system, comprising:
at least one storage device including a set of instructions; and
at least one processor configured to communicate with the at least one storage device, wherein when executing the set of instructions, the at least one processor is configured to direct the system to perform operations including:
obtaining first image data of a subject acquired by one or more first imaging devices;
determining, based on the first image data of the subject, pose information of the subject;
determining, based on the pose information of the subject, reference motion information of each of at least one of one or more detectors of a second imaging device; and
controlling a movement of each of the at least one of the one or more detectors based on the reference motion information for the second imaging device to acquire second image data of the subject.

2. The system of claim 1, wherein the first image data of the subject is acquired by the one or more first imaging devices during a scan of the subject or before the scan of the subject.

3. The system of claim 1, wherein the determining, based on the pose information of the subject, reference motion information of each of at least one of one or more detectors of a second imaging device includes:
obtaining initial motion information based on a first portion of one or more scanning parameters and the pose information; and
determining the reference motion information by optimizing the initial motion information based on a second portion of the one or more scanning parameters.

4. The system of claim 3, wherein determining the reference motion information by optimizing the initial motion information based on a second portion of the one or more scanning parameters includes:
determining one or more optimization objectives based on the second portion of the one or more scanning parameters; and
determining the reference motion information by optimizing the initial motion information based on the one or more optimization objectives using an optimization model, wherein the reference motion information satisfies the one or more optimization objectives.

5. The system of claim 4, wherein the optimization model is constructed based on an objective function, one or more constraints on at least one of reference path parameters of the reference motion information, the objective function being constructed based on the one or more optimization objectives and initial path parameters of the initial motion information, and
the optimizing the initial motion information based on the one or more optimization objectives using the optimization model includes:
optimizing the initial motion information by performing an iterative process based on the objective function, one or more constraints, and one or more optimization objectives to adjust the initial motion information.

6. The system of claim 4, wherein the optimization model is a first trained machine learning model, and optimizing the initial motion information based on the one or more optimization objectives includes:
inputting the initial motion information and the one or more optimization objectives into the first trained machine learning model to generate the reference motion information.

7. The system of claim 1, wherein the determining, based on the pose information of the subject, reference motion information of each of at least one of one or more detectors of a second imaging device includes:
determining, based on a scanning range of the subject and the pose information, a motion range of the each of at least one of one or more detectors; and
determining the reference motion information based on the motion range.

8. The system of claim 1, wherein the determining, based on the pose information of the subject, reference motion information of each of at least one of one or more detectors of a second imaging device includes:
determining, based on the pose information of the subject, the reference motion information using a second trained machine learning model.

9. The system of claim 8, wherein an input of the second trained machine learning model includes the pose information of the subject and at least one of medical image data acquired by a medical imaging device, physiological status information of the subject, or one or more scanning parameters of the subject, wherein the physiological status information of the subject is determined using the first imaging device.

10. The system of claim 1, wherein a count of the one or more detectors exceeds 1, and at least a portion of the one or more detectors are arranged along an axis of the second imaging device.

11. The system of claim 10, wherein controlling a movement of each of the at least one of the one or more detectors based on the reference motion information for the second imaging device acquiring second image data of the subject includes:
controlling the one or more detectors to move simultaneously based on the reference motion information of each of the one or more detectors to acquire the second image data of different portions of the subject during a same time period,
and the operations further include:
determining target image data of the subject based on the second image data of the different portions of the subject.

12. The system of claim 10, wherein the second imaging device includes multiple detector modules, each of the multiple detector modules includes at least one of the one or more detectors and a robot joint assembly for providing multiple movement freedoms of the one of the one or more detectors, and each of the multiple detector modules includes a first controller configured to control a component of the robot joint assembly to move based on the reference motion information.

13. The system of claim 1, wherein at least one of the one or more detectors is detachably connected with the second imaging device, and one of the one or more first imaging devices is arranged on one of the one or more detectors or on a ceiling of a scanning room where the second imaging device is located.

14. The system of claim 10, wherein the second image device includes multiple detector modules, each of the multiple detector modules includes one of the one or more detectors, a first controller, and a robot joint assembly for providing multiple movement freedoms of the one of the one or more detectors,
the second imaging device further includes a second controller in communication with the first controller of each of the multiple detector modules, wherein the second controller is configured to determine the reference motion information of each of the one or more detectors based on the pose information, the one or more scanning parameters, and position information of the one or more detectors, and transmit the reference motion information of each of the one or more detectors to a corresponding first controller.

15. The system of claim 1, wherein the pose information of the subject includes position information of a first contour of the subject, and the determining, based on the pose information of the subject, reference motion information of each of at least one of one or more detectors of a second imaging device includes:
obtaining a second contour of a couch where the subject is located;
determining a third contour by combining the first contour and the second contour; and
determining the reference motion information based on the third contour.

16. The system of claim 15, wherein the determining the reference motion information based on the third contour includes:
determining a minimum convex hull of the third contour; and
determining the reference motion information based on the minimum convex hull.

17. The system of claim 15, wherein the determining, based on the first image data of the subject, pose information of the subject includes:
controlling the one of the one or more detectors to move along an axis of the second imaging device for one of the one or more first imaging device arranged on the one of the one or more detectors to acquire first sub-image data is at each of different positions;
determining a first sub-contour of a portion of the subject based on the first sub-image data of the subject; and
determining the first contour based on first sub-contours of different portions of the subject.

18. The system of claim 17, wherein the second contour includes a first sub-contour and a second sub-contour associated with a surface of the couch where the subject is located, and the determining a third contour by combining the first contour and the second contour includes: combining the first sub-contour of the second contour and the first contour via the second sub-contour of the second contour.

19. A method implemented on a computing device having at least one processor and at least one storage medium including a set of instructions for processing service requests received from a requester terminal, comprising:
obtaining first image data of a subject acquired by one or more first imaging devices;
determining, based on the first image data of the subject, pose information of the subject;
determining, based on the pose information of the subject, reference motion information of each of at least one of one or more detectors of a second imaging device; and
controlling a movement of each of the at least one of the one or more detectors based on the reference motion information for the second imaging device to acquire second image data of the subject.

20. A non-transitory computer readable medium, comprising executable instructions that, when executed by at least one processor, direct the at least one processor to perform a method comprising:
obtaining first image data of a subject acquired by one or more first imaging devices;
determining, based on the first image data of the subject, pose information of the subject;
determining, based on the pose information of the subject, reference motion information of each of at least one of one or more detectors of a second imaging device; and
controlling a movement of each of the at least one of the one or more detectors based on the reference motion information for the second imaging device to acquire second image data of the subject.
